(19) United States
(12) Patent Application Publication
(10) Pub. No.: US 2014/0329755 A1
(43) Pub. Date: Nov. 6, 2014

(54) COMBINATION THERAPY FOR THE TREATMENT OF ARRHYTHMIAS OR HEART FAILURE

(71) Applicant: Gilead Sciences, Inc., Foster City, CA (US)

(72) Inventors: Luiz Belardinelli, Palo Alto, CA (US); Peidong Fan, San Mateo, CA (US); Faquan Liang, San Francisco, CA (US); Lina Yao, San Mateo, CA (US)

(73) Assignee: Gilead Sciences, Inc., Foster City, CA (US)

(21) Appl. No.: 14/266,558

(22) Filed: Apr. 30, 2014

Related U.S. Application Data
(60) Provisional application No. 61/818,297, filed on May 1, 2013.

(51) Int. Cl. A61K 38/16; A61K 31/553; A61K 31/437; A61K 31/18; A61K 31/495

(52) U.S. Cl. CPC A61K 38/16; A61K 31/18; A61K 31/495; A61K 31/437; A61K 31/553
USPC 514/16.4; 514/604; 514/252.12; 514/303; 514/211.05

(57) ABSTRACT

The present invention relates to method of treating arrhythmias or heart failure comprising co-administration of a late I_{Na} inhibitor and a CAMK II inhibitor.

### CROSS-REFERENCE TO RELATED APPLICATIONS

This application claims benefit under 35 U.S.C. §119(e) to U.S. Provisional Application Ser. No. 61/818,297, filed on May 1, 2013, the entirety of which is incorporated herein by reference

### FIELD OF THE INVENTION

The present invention relates to a combination therapy for the treatment of arrhythmias.

### BACKGROUND

Atrial fibrillation (AF) is the most prevalent arrhythmia, the incidence of which increases with age. It is estimated that 8% of all people over the age of 80 experience this type of abnormal heart rhythm and AF accounts for one-third of hospital admissions for cardiac rhythm disturbances. Over 2.2 million people are believed to have AF in the United States alone (Fuster et al., Circulation 2006 114: e257-354). Although atrial fibrillation is often asymptomatic it may cause palpitations or chest pain. Prolonged atrial fibrillation often results in the development of congestive heart failure and/or stroke. Heart failure develops as the heart attempts to compensate for the reduced cardiac efficiency while stroke may occur when thrombi form in the atria, pass into the blood stream and lodge in the brain. Pulmonary emboli may also develop in this manner.

Heart failure is a clinical syndrome characterized by the failure of the heart to pump sufficient blood to meet the body's systemic demands. Heart failure may be subdivided into systolic or diastolic heart failure. Systolic heart failure results from reduced cardiac contractility. Diastolic heart failure results from impaired cardiac relaxation and abnormal ventricular filing. Heart failure is a common syndrome especially in older adults. Currently, approximately 2% of the U.S population is afflicted with heart failure. Although the survival rate for heart failure has improved with reperfusion therapy, most reperfused patients have some residual left ventricle systolic dysfunction which can lead to heart failure. (Heart Failure, Robert Hobbs and Andrew Boyle, https://www.clevelandclinicmeded.com/medicalpubs/diseasemanagement/cardiology/heart-failure; visited Apr. 28, 2014).

Cellular sodium and calcium overload caused by enhancement of the late Na⁺ current (late I_{Na}) may play a critical role in cardiac arhythmogenesis and diastolic dysfunction. (Katra R P, Laurita K R: Cellular mechanism of calcium-mediated triggered activity in the heart, Circ Res 96: 535-542, 2005). Voltage gated Na⁺ channels are activated upon depolarization of the myocyte membrane, which leads to a rapid and brief influx of Na⁺ that generates the upstroke of the cardiac action potential (AP). Normally, Na⁺ channels inactivate within a few milliseconds after depolarization. However, during pathological conditions such as heart failure, ischemia/hypoxia and oxidative stress, some Na⁺ channels remain open, creating a small but persistent influx of Na⁺ throughout the plateau of the AP. This current is referred to as late I_{Na} and it reduces repolarization reserve and prolongs the duration of the AP. Enhancement of late I_{Na} facilitates occurrences of early afterdepolarizations (EADs) and Na⁺/Ca²⁺ loading that leads to delayed afterdepolarizations (DADs) in both atrial and ventricular myocadium. (Noble D, Noble P J: Late sodium current in the pathophysiology of cardiovascular disease: consequences of sodium-calcium overload, Heart 2006; 92:iv1-5; See also, Sossalla S, et al.; Ranolazine improves diastolic dysfunction in isolated myocardium from failing human hearts—role of late sodium current and intracellular ion accumulation. J Mol Cell Cardiol. 2008; 45: 32-43. Enhancement of late I_{Na} is associated with increases of triggered and reentrant arrhythmias and torsades de pointes tachycardia. In guinea pig isolated atrial myocytes, enhancement of late I_{Na} has been found to cause EADs, DADs, and spontaneous diastolic depolarization, and in atrial myocytes isolated from patients with heart failure.

An increase in late I_{Na} may also lead to activation of the Ca²⁺/calmodulin-dependent protein kinase (CaMKII). A late I_{Na}-induced Na⁺ influx into myocytes increases calcium influx via the reverse mode of the sodium/calcium exchanger (NCX). (Nattel S., Dobrev D. The multidimensional role of calcium in atrial fibrillation pathophysiology: mechanistic insights and therapeutic opportunities. Eur Heart J. 2012; 33:1870-7). When intracellular Ca²⁺ levels are increased, Ca²⁺ binds to calmodulin (CaM). The Ca²⁺/CaM complex can activate Ca²⁺/calmodulin-dependent protein kinase (CaMKII), a multifunctional serine/threonine protein kinase (Hudmon A, Schulman H. Structure-function of the multifunctional Ca2+/calmodulin-dependent protein kinase II. Biochem J. 2002; 364:593-611). An increase in CaMKII activity has been implicated as a contributory cause of cardiac arrhythmias and diastolic heart failure (Ai X, Curran J W, Shannon T R, Bers D M, and Pogwizd S M: Ca2+/calmodulin-dependent protein kinase modulates cardiac ryanodine receptor phosphorylation and sarcoplasmic reticulum Ca2+ leak in heart failure. Circ Res. 2005; 97:1314-22).

There are four isoforms of CaMKII: α, β, γ and δ. CaMKIIδ is the predominant isoform expressed in the heart. Activated CaMKIIδ phosphorylates a number of key Ca²⁺ handling proteins in cardiac myocytes, including ryanodine receptor 2 (RyR2), the sarcoplasmic/endoplasmic reticulum Ca²⁺-ATPase (SERCA), phospholamban (PLB) and the L-type Ca²⁺ channel (LTCC). CaMKIIδ also phosphorylates the cardiac Na⁺ channel Naᵥ1.5, leading to increased late I_{Na} (26-31). Mutational analysis revealed that CaMKII directly phosphorylates Naᵥ1.5 at serine 571 to enhance late I_{Na} and increase myocyte susceptibility to afterdepolarizations (Hund T J, et al: A β(IV)-spectrin/CaMKII signaling complex is essential for membrane excitability in mice. J Clin Invest; 2010; 120:3508-19). Inhibition of late I_{Na} reverses diastolic heart failure.

In view of the prevalence of arrhythmias and heart failure and the need to provide improved treatment outcomes for patients suffering from or presenting with arrhythmias or heart disease, there is a need to discover new effective treatments for heart disease, particularly atrial fibrillation and/or heart failure.

### SUMMARY OF THE INVENTION

The present disclosure is based on the discovery that co-administration of a CAM kinase II (CAMKII) inhibitor and a late I_{Na} inhibitor provides superior and synergistic atrial rate and/or rhythm control compared to the effect observed with administration of either agent alone. The ability to control the atrial rate and/or the atrial rhythm is useful for treating and/or preventing atrial fibrillation and/or atrial flutter in patients. Atrial rate and rhythm control is also useful for treating a variety of other cardiac conditions, which are described herein. In particular the discovery is useful for the prevention and/or treatment of heart failure (including systolic and diastolic heart failure) or the prevention of a second heart condition. It is contemplated that the co-administration is useful when the CAMK II inhibitor is administered in a therapeutically effective dose and the late I_{Na} inhibitor is administered in a therapeutically effective dose. It is further contemplated that the CAMK II inhibitor and the late I_{Na} inhibitor may be co-administered in an amount less than their respective previously used, disclosed, or approved therapeutic doses due to their combined synergistic effect.

Accordingly, in one aspect, the present disclosure provides a method for treating arrhythmias and/or heart failure in a human patient comprising administering to the patient a therapeutically effective amount of a CAMK II inhibitor and a therapeutically effective amount of a late I_{Na} inhibitor.

In another aspect, the present disclosure provides a method for treating heart failure in a human patient in need thereof, comprising administering to the patient a therapeutically effective amount of a CAMK II inhibitor and a therapeutically effective amount of a late I_{Na} inhibitor.

In another aspect, the present disclosure provides a method for treating atrial fibrillation or atrial flutter in a human patient in need thereof, comprising administering to the patient a therapeutically effective amount of a CAMK II inhibitor and a therapeutically effective amount of a late I_{Na} inhibitor.

In another aspect, the present disclosure provides a pharmaceutical composition comprising a therapeutically effective amount of a CAMK II inhibitor or a pharmaceutically acceptable salt thereof, a therapeutically amount of a late I_{Na} inhibitor or a pharmaceutically acceptable salt thereof, and a pharmaceutically acceptable carrier.

In another aspect, the disclosure is directed to a pharmaceutical formulation comprising a therapeutically effective amount of a CAMK II inhibitor or pharmaceutically acceptable salt thereof, a therapeutically effective amount of ranolazine, and a pharmaceutically acceptable carrier.

In another aspect, the disclosure is directed to a pharmaceutical formulation comprising a therapeutically effective amount of a CAMK II inhibitor or pharmaceutically acceptable salt thereof, a therapeutically effective amount of GS-967 (6-(4-(trifluoromethoxy)phenyl)-3-(trifluoromethyl)-[1,2,4]thazolo[4,3-a]pyridine), and a pharmaceutically acceptable carrier.

In another aspect, the disclosure is directed to a pharmaceutical formulation comprising a therapeutically effective amount of a CAMKII inhibitor selected from KN-93 and AIP, or pharmaceutically acceptable salt thereof, a therapeutically effective amount of late INa inhibitor selected from ranolazine and GS-967, and a pharmaceutically acceptable carrier.

In another aspect, the disclosure is directed to a pharmaceutical formulation comprising a therapeutically effective amount of a CAMKII inhibitor selected from KN-93 and AIP, or pharmaceutically acceptable salt thereof, a therapeutically effective amount of late Na inhibitor selected from compound of Formula I, Formula II or Formula III disclosed herein, and a pharmaceutically acceptable carrier.

In yet another aspect, the present disclosure provides the use of a therapeutically effective amount of a CAMK II inhibitor and a therapeutically effective amount of a late I_{Na} inhibitor for the manufacture of a medicament for treating arrhythmias and/or heart failure.

In yet another aspect, the present disclosure provides the use of a therapeutically effective amount of a CAMK II inhibitor selected from AIP and KN-93 and a therapeutically effective amount of a late I_{Na} inhibitor selected from ranolazine, GS-967, or a compound of formula I, formula II and formula III disclosed herein for the manufacture of a medicament for treating arrhythmias or heart failure.

In yet another aspect, the present disclosure provides the use of a therapeutically effective amount of a CAMK II inhibitor and a therapeutically effective amount of a late I_{Na} inhibitor for use in therapy.

Applicants have discovered a synergistic effect arising from the combination of a CAMK II inhibitor and a late I_{Na} inhibitor. The combination of a therapeutically effective CAMK II inhibitor and a late I_{Na} inhibitor based on the synergistic findings herein disclosed is expected to provide additional beneficial treatment effect that is more than the effect of either agent alone or the mathematically combined additive effect of both agents. Thus, applicants have disclosed a new and important tool in the treatment of arrhythmias (particularly atrial fibrillation) and/or heart failure (systolic or diastolic).

In another aspect, the disclosure is directed to a method for modulating ventricular and/or atrial rate in a patient in need thereof, said method comprising co-administering to the patient therapeutic amounts of a late I_{Na} inhibitor, e.g. ranolazine; and a CAMK II inhibitor, e.g. KN-93, or pharmaceutically acceptable salt or salts thereof.

In another aspect, the disclosure is directed to a method for modulating rhythm control in a patient in need thereof, said method comprising co-administering to the patient therapeutic amounts of a late I_{Na} inhibitor, e.g. ranolazine; and a CAMK II inhibitor, e.g. KN-93, or pharmaceutically acceptable salt or salts thereof.

In another embodiment, the disclosure is directed to a method for treating atrial fibrillation comprising co-administration of a therapeutically effective amount of a CAMK II inhibitor and ranolazine or GS-967, or pharmaceutically acceptable salt or salts thereof, to a patient in need thereof.

In another embodiment, the disclosure is directed to a method for the treatment of atrial fibrillation comprising co-administering a therapeutically effective amount of a CAMK II inhibitor and a late I_{Na} inhibitor or pharmaceutically acceptable salt or salts thereof wherein the therapeutically effective amount of a CAMK II inhibitor and therapeutically effective amount of a late I_{Na} inhibitor are co-administered separately.

In another aspect, the disclosure is directed to a method for treating heart failure in a patient in need thereof, said method comprising co-administering to the patient therapeutic amounts of a late I_{Na} inhibitor, e.g. ranolazine; and a CAMK II inhibitor, e.g. KN-93, or pharmaceutically acceptable salt or salts thereof.

In one embodiment, the disclosure is directed to a method for treating heart failure comprising co-administration of a therapeutically effective amount of a CAMK II inhibitor and ranolazine or GS-967, or pharmaceutically acceptable salt or salts thereof, to a patient in need thereof.

In another embodiment, the disclosure is directed to a method for the treatment of heart failure comprising co-administering a therapeutically effective amount of a CAMK II inhibitor and a late I_{Na} inhibitor or pharmaceutically acceptable salt thereof wherein the therapeutically effective amount of a CAMK II inhibitor and therapeutically effective amount of a late I_{Na} inhibitor are co-administered separately. The therapeutically effective amount of a CAMK II inhibitor and therapeutically effective amount of a late I_{Na} inhibitor may also be administered simultaneously or sequentially.

In another embodiment, the disclosure is directed to a method for the treatment of atrial fibrillation or heart failure comprising administering a therapeutically effective amount of a CAMK II inhibitor and a late I_{Na} inhibitor or pharmaceutically acceptable salt or salts thereof, wherein the therapeutically effective amount of the CAMK II inhibitor and therapeutically effective amount of the late I_{Na} inhibitor are administered together in a combined dosage form.

In another embodiment, the disclosure is directed to a method for the treatment of atrial fibrillation or heart failure comprising administering a therapeutically effective amount of a CAMK II inhibitor and a late I_{Na} inhibitor or pharmaceutically acceptable salt or salts thereof, wherein the therapeutically effective amount of a CAMK II inhibitor and therapeutically effective amount of a late I_{Na} inhibitor are co-administered in a solid fixed dose combination.

In another embodiment, the present disclosure provides a pharmaceutical composition comprising a therapeutically effective amount of a CAMK II inhibitor and therapeutically effective amount of a late I_{Na} inhibitor and a pharmaceutically acceptable carrier.

### BRIEF DESCRIPTION OF THE DRAWINGS

As used throughout the Figures, the term “RAN” refers to ranolazine.

FIG. 1 shows the combination effect of a late I_{NA} inhibitor and a CaMKII inhibitor on ATX-II-induced AF.

FIG. 2 shows the effect of RAN or GS-967 in combination with AIP on ATX-II-induced CaMKII activity.

FIG. 3 shows that inhibition of late I_{Na} and CaMKII prevents ATX-II-induced diastolic Ca²⁺ increase in isolated rat atrial myocytes.

### DETAILED DESCRIPTION OF THE INVENTION

### Definitions and General Parameters

As used in the present specification, the following words and phrases are generally intended to have the meanings as set forth below, except to the extent that the context in which they are used indicates otherwise.

It is to be noted that as used herein and in the claims, the singular forms “a,” “an” and “the” include plural referents unless the context clearly dictates otherwise. Thus, for example, reference to “a pharmaceutically acceptable carrier” in a composition includes two or more pharmaceutically acceptable carriers, and so forth.

“Comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. The phrase “consisting of” shall mean excluding more than trace elements of other ingredients and substantial method steps for administering the compositions of this invention. Embodiments defined by each of these transition terms are within the scope of this invention.

As used herein the term “invention” and “disclosure” are synonymous as it relates to the subject matter herein disclosed and/or claimed.

The term “therapeutically effective amount” refers to that amount of a late I_{Na} inhibitor or a CAMK II inhibitor compound, such as for example ranolazine, GS-967, AIP, or KN-93 that is sufficient to effect treatment as defined or disclosed herein, when administered to a human patient in need of such treatment. The therapeutically effective amount may vary depending upon the pharmacological activity of the therapeutic agent being used, the severity of the patient's disease state, age, physical condition, existence of other disease states, and nutritional status of the patient. Additionally, other medication the patient may be receiving may affect the determination of the therapeutically effective amount of the therapeutic agent to administer. In some embodiments, the term “therapeutically effective amount” refers to a synergistically effective amount or synergistically therapeutic amount.

“Synergistic” means that the therapeutic effect of the CAMK II inhibitor when administered in combination as described herein with a late I_{Na} inhibitor (or vice-versa) is greater than the predicted additive therapeutic effects of CAMK II inhibitor and late I_{Na} inhibitor when administered alone. The term “synergistically therapeutic amount” or “synergistically effective amount” refers to a less than standard therapeutic amount of one or both drugs, meaning that the amount required for the desired effect is lower than when the drug is used alone. A synergistically therapeutic amount also includes when one drug is given at a standard therapeutic dose and another drug is administered in a less than standard therapeutic dose. For example, the late I_{Na} inhibitor could be given in a therapeutic dose and the CAMK II inhibitor could be given in a standard or less than standard therapeutic dose to provide a synergistic result.

The term “treatment” or “treating” means any administration of a composition according to the present disclosure to a mammal, e.g. a human, suffering a disease or condition for the purpose of 1) preventing or protecting against the disease or condition, i.e. causing the clinical symptoms not to develop; 2) inhibiting the disease or condition, i.e. arresting or suppressing the development of clinical symptoms; and/or 3) relieving the disease or condition i.e. causing the regression of clinical symptoms.

The term “susceptible” refers to a patient who has had at least one occurrence of the indicated condition or is predisposed genetically or otherwise to having an occurrence of the indicated condition.

As used herein, “pharmaceutically acceptable carrier” includes any and all excipients, including solvents, dispersion media, coatings, antibacterial and antifungal agents, isotonic and absorption delaying agents and the like that are found suitable for the purpose of formulating the combined dosage form of the invention disclosed herein. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Atrial fibrillation” or “AF” occurs when the heart's two upper chambers (the right and left atria) quiver instead of beating and contracting rhythmically. Electrocardiographically, AF is characterized by a highly disorganized atrial electrical activity that often results in fast beating of the heart's two lower chambers (the right and left ventricles). Symptoms experienced by patients with AF include palpitation, fatigue, and dyspnea (shortness of breath).

There are three known types of AF based on the presentation and duration of the arrhythmia: a) Paroxysmal AF: recurrent AF (>2 episodes) that starts and terminates spontaneously within 7 days (paroxysmal AF starts and stops spontaneously); b) Persistent AF: sustained AF that lasts longer than 7 days or requires termination by pharmacologic or electrical cardioversion (electrical shock); and c) Permanent AF: long standing AF (for >1 year duration) in which normal sinus rhythm cannot be maintained even after treatment, or when the patient and physician have decided to allow AF to continue without further efforts to restore sinus rhythm.

“Atrial flutter” is an abnormal heart rhythm that occurs in the atria of the heart. When it first occurs, it is usually associated with a fast heart rate or tachycardia (230-380 beats per minute (bpm)), and falls into the category of supra-ventricular tachycardia. While this rhythm occurs most often in individuals with cardiovascular disease (e.g. hypertension, coronary artery disease, and cardiomyopathy), it may occur spontaneously in people with otherwise normal hearts. It is typically not a stable rhythm, and frequently degenerates into atrial fibrillation (AF).

Both “electrical and structural remodeling” contribute to the pathogenesis of AF. Electrical triggers (after potentials) and arrhythmogenic substrate (re-entry) are two main causes for the initiation and maintenance of AF. “Electrical remodeling” is caused by malfunctioning of ion channels (mainly sodium, calcium, and potassium channels). “Structural remodeling” is caused by proliferation and differentiation of fibroblasts into myofibroblasts and enhanced connective tissue deposition. Structural remodeling results in the electrical dissociation between cardiac muscle bundles and heterogeneity in the electrical conduction in the atrium. Thus, inflammation and/or fibrosis of atrial tissue create a milieu conducive for AF. The electrical and structural remodeling of the atria leads to the perpetuation of AF. Hence, “AF begets AF”. Prolonged episodes of AF frequently cause mechanical dysfunction of the atrium resulting in adverse hemodynamic consequences and may contribute to heart failure.

“Ventricular fibrillation” occurs when the heart beats with rapid, erratic electrical impulses which cause pumping chambers in the heart (i.e. the ventricles) to quiver uselessly, rather than pump blood. Ventricular fibrillation requires immediate medical attention as blood pressure plummets, cutting off blood supply to vital organs. A person with ventricular fibrillation will collapse within seconds and soon will not be breathing or have a pulse. Symptoms include chest pain, rapid heartbeat (tachycardia), dizziness, nausea, shortness of breath, and loss of consciousness or fainting. It is not always known what causes ventricular fibrillation, but most cases of ventricular fibrillation begin as a rapid heartbeat called “ventricular tachycardia” or “VT”.

“Torsades de pointes (or TdP) ventricular tachycardia” refers to a specific variety of ventricular tachycardia that exhibits distinct characteristics on the electrocardiogram (ECG). The ECG reading in torsades demonstrates a rapid, polymorphic ventricular tachycardia with a characteristic twist of the QRS complex around the isoelectric baseline. It is also associated with a fall in arterial blood pressure, which can produce fainting. Although “torsades de pointes” is a rare ventricular arrhythmia, it can degenerate into “ventricular fibrillation”, which will lead to sudden death in the absence of medical intervention. Torsades de pointes is associated with long QT syndrome, a condition whereby prolonged QT intervals are visible on the ECG. Long QT intervals predispose the patient to an R-on-T phenomenon, where the R wave representing ventricular depolarization occurs simultaneously to the relative refractory period at the end of repolarization (represented by the latter half of the T-wave). An R-on-T can initiate torsades. Long QT syndrome can either be inherited as congenital mutations of ion channels carrying the cardiac impulse/action potential or acquired as a result of drugs that block these cardiac ion currents.

Common causes for torsades de pointes include diarrhea, hypomagnesemia, and hypokalemia. It is commonly seen in malnourished individuals and chronic alcoholics. Drug interactions such as erythromycin or moxifloxacin, taken concomitantly with inhibitors like nitroimidazole, dietary supplements, and various medications like methadone, lithium, tricyclic antidepressants or phenothiazines may also contribute. It can also be the side effect of some anti-arrhythmic medications such as sotalol, procainamide, and quinidine. Factors that are associated with an increased tendency toward torsades de pointes include: class IA antiarrhythmics, class III antiarrhythmics, hypomagnesemia, hypokalemia, hypocalcemia, hypoxia, acidosis, heart failure, left ventricular hypertrophy, slow heart rate, female gender, hypothermia, subarachnoid hemorrhage.

“AV conduction” or “atrioventricular conduction” is the forward conduction of the cardiac impulse from the atria to ventricles via the “atrioventricular node” or “AV node”, represented in an electrocardiogram by the P-R interval. The AV node is a part of electrical control system of the heart that electrically connects atrial and ventricular chambers and coordinates heart rate. The AV node is an area of specialized tissue between the atria and the ventricles of the heart, specifically in the posteroinferior region of the inter-atrial septum near the opening of the coronary sinus, which conducts the normal electrical impulse from the atria to the ventricles. “AV conduction” during normal cardiac rhythm occurs through two different pathways: the first has a slow conduction velocity but shorter refractory period, whereas the second has a faster conduction velocity but longer refractory period.

The term “modulate” means to increase or decrease or otherwise provide control.

“Modulating ventricular and/or atrial rate” has been shown to significantly improve AF. Typically, this has been accomplished with the use of a pacemaker, where the pacemaker detects the atrial beat and after a normal delay (0.1-0.2 seconds) triggers a ventricular beat, unless it has already happened—this can be achieved with a single pacing lead with electrodes in the right atrium (to sense) and ventricle (to sense and pace). The “atrial rate” is specific to the rate (measured in beats per unit time) of only the atrial beat. Pacemakers can also monitor and modulate the ventricular and/or atrial rhythm. The “ventricular and/or atrial rhythm” refers to the beat-to-beat time period of either the ventricular beat or the atrial beat.

As used herein the terms “CAMK II inhibitor”, “CAMK inhibitor”, and “CAM Kinase inhibitor” are synonymous.

“Co-administering” or “co-administration” refers to the administration of two or more therapeutic agents together at one time or within a given time frame. The two or more therapeutic agents can be coformulated into a single dosage form or “combined dosage unit”, or formulated separately and subsequently combined into a combined dosage unit, typically for intravenous administration or oral administration.

“Intravenous administration” is the administration of substances directly into a vein, or “intravenously”. Compared with other routes of administration, the intravenous (IV) route is the fastest way to deliver fluids and medications throughout the body. An infusion pump can allow precise control over the flow rate and total amount delivered, but in cases where a change in the flow rate would not have serious consequences, or if pumps are not available, the drip is often left to flow simply by placing the bag above the level of the patient and using the clamp to regulate the rate. Alternatively, a rapid infuser can be used if the patient requires a high flow rate and the IV access device is of large enough diameter to accommodate it. This is either an inflatable cuff placed around the fluid bag to force the fluid into the patient or a similar electrical device that may also heat the fluid being infused. When a patient requires medications only at certain times, intermittent infusion is used, which does not require additional fluid. It can use the same techniques as an intravenous drip (pump or gravity drip), but after the complete dose of medication has been given, the tubing is disconnected from the IV access device. Some medications are also given by IV push or bolus, meaning that a syringe is connected to the IV access device and the medication is injected directly (slowly, if it might irritate the vein or cause a too-rapid effect). Once a medicine has been injected into the fluid stream of the IV tubing there must be some means of ensuring that it gets from the tubing to the patient. Usually this is accomplished by allowing the fluid stream to flow normally and thereby carry the medicine into the bloodstream; however, a second fluid injection is sometimes used, as a “flush”, following the injection to push the medicine into the blood stream more quickly.

“Oral administration” is a route of administration where a substance is taken through the mouth, and includes buccal, sublabial and sublingual administration, as well as enteral administration and that through the respiratory tract, unless made through e.g. tubing so the medication is not in direct contact with any of the oral mucosa. Typical form for the oral administration of therapeutic agents includes the use of tablets or capsules.

A “sustained release formulation” is a formulation which is designed to slowly release a therapeutic agent or agents in the body over an extended period of time, whereas an “immediate release formulation” is a formulation which is designed to quickly release a therapeutic agent in the body over a shortened period of time. In some cases the immediate release formulation may be coated such that the therapeutic agent is only released once it reached the desired target in the body (e.g. the stomach).

### Methods

One object of the disclosure is to provide a method for treating arrhythmias, particularly atrial fibrillation comprising co-administration of a therapeutically effective amount of a CAMK II inhibitor or salt thereof and a therapeutically effective amount of a late I_{Na} inhibitor or salt thereof to a human patient in need thereof. Another object of the present disclosure to provide a method for treating heart failure (systolic or diastolic) comprising co-administration of a therapeutically effective amount of a CAMK II inhibitor or salt thereof and a therapeutically effective amount of a late I_{Na} inhibitor or salt thereof to a human patient in need thereof. Prior to applicants' disclosure, synergistic beneficial effect of treatment with a CAMK II inhibitor and a late I_{Na} inhibitor was unknown, and not suggested. Applicants' have discovered a beneficial synergistic effect arising from the combination of a CAMK II inhibitor, e.g. KN-93, and a late I_{Na} inhibitor, e.g. ranolazine.

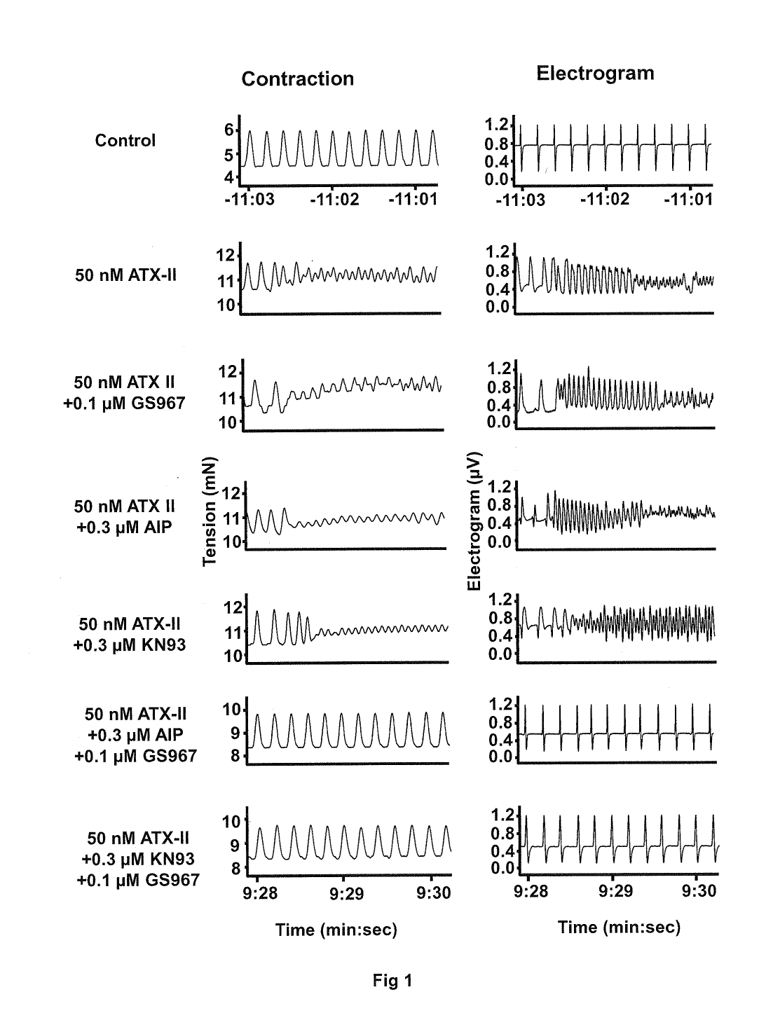

Applicants observed that inhibition of late I_{Na} prevented ATX-II induced arrhythmias and diastolic dysfunction in isolated rat right atria (see FIG. 1). Specifically, the late I_{Na} inhibitors GS-967 and ranolazine demonstrated IC₅₀ values of 1.29 μM and 14.8 μM respectively. Similarly, applicants observed that inhibition of late I_{Na} prevented ATX-II induced AF in isolated rat right atria.

CAMK II related experiments showed for example that inhibition of CAMK II with either AIP or KN-93 prevented ATX-II induced arrhythmias and diastolic dysfunction in rat right atria. Similar to the effect of late Ina inhibitors, applicants observed that inhibition of CAMK II prevented ATX-II induced AF in isolated rat right atria.

Surprisingly, applicants have discovered that the combination of a late I_{Na} inhibitor and a CAMK inhibitor prevented ATX-II induced arrhythmias. More surprisingly, the combined effect of a CAMK II inhibitor and a late I_{Na} inhibitor was synergistic. For example, the calculated combined effect of 3 μM ranolazine and 0.3 μM KN-93 is 47.9+−5.6% whereas the observed combined effect was 83.3+−3.0% inhibition.

Applicants believe the practice of the disclosure herein will provide greater antiarrythmic benefit to patients and reduce the treatment cost burden of arrhythmia.

### Active Ingredients and Compositions

### CAMK II inhibitors

Autocamtide inhibitory peptide (AIP) is a selective and potent calmodulin-dependent protein kinase II (CAMK II) inhibitor. It is commercially available, for example, from Tocris BioSciences, Bristol, UK.

KN-93 is a potent, cell permeable inhibitor of CAMK II. It is also a direct open channel blocker of voltage gated potassium channels independent of CAMK II inhibition. KN-93 is represented by the structure below.

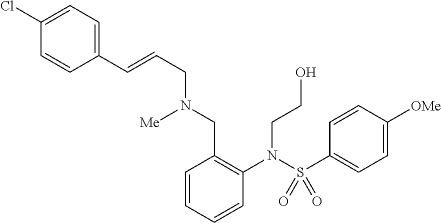 [SMILES: COc1ccc(S(=O)(=O)N(CCO)c2ccccc2CN(C)C/C=C/c2ccc(Cl)cc2)cc1]

KN-93 (CAS #139298-40-1) is commercially available from numerous sources including for example, Tocris Biosciences Ltd, (UK), Sigma Aldrich Corporation (USA), CalBiochem (USA) and Santa Cruz Biotech, (USA). KN-93 may also be prepared as described in the literature. See for example, Claudio Bruno et al., Microwave-Assisted Synthesis of KN-93, a Potent and Selective Inhibitor of Ca²⁺/Calmoduline-Dependent Protein Kinase II, Synthesis 2010 (24) 4193-4198.

### Ranolazine

“Ranolazine” or “RAN” is described in U.S. Pat. No. 4,567,264. It refers to the chemical compound (±)—N-(2,6-dimethylphenyl)-4-[2-hydroxy-3-(2-methoxyphenoxy)-propyl]-1-piperazineacetamide. Pharmaceutically acceptable salts of ranolazine are accordingly, for example, as Ran HCl or ranolazine HCl. In its dihydrochloride salt form, ranolazine is represented by the formula:

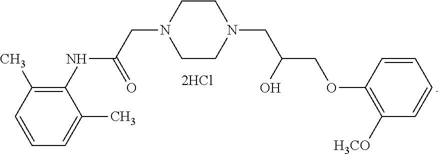 [SMILES: COc1ccccc1OCC(O)CN1CCN(CC(=O)Nc2c(C)cccc2C)CC1]

Methods of preparing ranolazine are known to one of skill in the art.

A preferred method of making ranolazine is disclosed in U.S. Patent Application Publication Number 2006/0177502 (the entirety of which is incorporated by reference herein). U.S. Patent Application Publication Number 2006/0177502 discloses oral sustained release dosage forms in which the ranolazine is present in 35-50%, preferably 40-45% ranolazine. In one embodiment the ranolazine sustained release formulations of the invention include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3-6%. Suitable pH independent binders include, but are not limited to, hydroxypropylmethylcellulose (HPMC), for example Methocel® E10M Premium CR grade HPMC or Methocel® E4M Premium HPMC. Suitable pharmaceutically acceptable excipients include magnesium stearate and microcrystalline cellulose (Avicel® pH101).

A particularly preferred method of preparing sustained release formulation of ranolazine is disclosed in U.S. Pat. No. 6,503,911, the entirety of which is incorporated herein by reference.

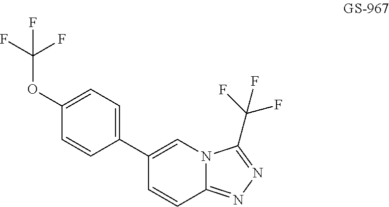 [SMILES: FC(F)(F)Oc1ccc(-c2ccc3nnc(C(F)(F)F)n3c2)cc1]

GS-967 is 6-[4-(trifluoromethoxy)phenyl]-3-(trifluoromethyl)[1,2,4]triazolo[4,3-a]pyridine. It is a potent and selective inhibitor of late I_{Na}. GS-967 is also known as GS-458967. The effectiveness of GS-967 to suppress ventricular arrhythmias has been reported previously. GS967 inhibited Anemonia sulcata toxin II (ATX-II)-induced late I_{Na} in ventricular myocytes and isolated hearts with IC₅₀ values of 0.13 μM and 0.21 μM, respectively. Reduction of peak I_{Na} by GS967 was minimal at a holding potential of −120 mV but increased at −80 mV. GS967 did not prolong action potential duration or the QRS interval. QRS intervals are known to persons of ordinary skill in the art. GS967 prevented and reversed proarrhythmic effects (afterdepolarizations and torsades de pointes) of the late I_{Na} enhancer ATX-II and the I_{Kr} inhibitor E-4031 in isolated ventricular myocytes and hearts. GS-967 significantly attenuated the proarrhythmic effects of methoxamine+clofilium and suppressed ischemia-induced arrhythmias. GS-967 was more potent and effective to reduce late I_{Na} and arrhythmias than either flecainide or ranolazine. See Bellardinelli et al; A novel, potent, and selective inhibitor of cardiac late sodium current suppresses experimental arrhythmias, J Pharmacol Exp Ther. 2013 January; 344(1):23-32.

Methods of preparing GS-967 are disclosed in WO 2011/014462 (pages 75-76), which is incorporated herein in its entirety. Further, an embodiment of the present invention is the use of any one of the late I_{Na} inhibitor compounds disclosed in WO 2011/014462 in combination with a CAM Kinase inhibitor according to the present disclosure.

### Other Late I_{Na} Inhibitors

In one embodiment, the present disclosure provides a method for treating atrial fibrillation, atrial flutter or heart failure comprising administering a therapeutically effective amount of a CAMK inhibitor (e.g. KN-93 or AIP) in combination with a compound of formula I:

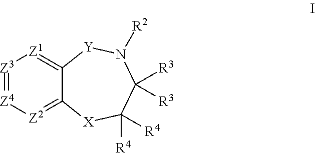

wherein:
Z¹ and Z² are each independently selected from the group consisting of CR⁷ and N;
Z³ and Z⁴ are each independently selected from the group consisting of CR⁷, C-Q-R¹ and N, provided that one of Z³ and Z⁴ is C-Q-R¹ and the other of Z³ and Z⁴ is CR⁷ or N and further provided that only one of Z¹, Z² and Z⁴ is N;
X is —O— or —NR⁶—;
Y is —C(O)—, —C(R¹¹)₂— or —S(O)₂—;
Q is a covalent bond, —O—C₀₋₂ alkylene, —NR¹¹—C₀₋₂ alkylene, C₂ alkylene, C₂ alkenylene or C₂ alkynylene;
R¹ is aryl, cycloalkyl, cycloalkenyl, heterocyclyl or heteroaryl;
   wherein said aryl, cycloalkyl, cycloalkenyl, heterocyclyl or heteroaryl are optionally substituted with one, two or three substituents independently selected from the group consisting of halo, —NO₂, —CN, —SF₅, —Si(CH₃)₃, —O—R²⁰, —S—R²⁰, —C(O)—R²⁰, —C(O)—OR²⁰, N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, cycloalkyl, aryl, heteroaryl and heterocyclyl; and
   wherein said C₁₋₆ alkyl, C₂₋₄ alkenyl, C₂₋₄ alkynyl, cycloalkyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, cycloalkyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵, -L-R⁵, alkylene-R⁵, —C₁₋₆ alkylene-L-R⁵ or —C₁₋₆ alkylene-L-C₁₋₆ alkylene-R⁵;
   wherein each —C₁₋₆ alkylene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, C(O)—N(R²⁰)(R²²), —N(R²⁰)—S(O)₂—R²⁰, cycloalkyl, aryl, heteroaryl or heterocyclyl; and
      wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;
L is —O—, —S—, —C(O)—, —NHS(O)₂—, —S(O)₂NH—, —C(O)NH—, or —NHC(O)—; provided that when Y is —C(R¹¹)₂—, then R² is -L-R⁵, -L-C₁₋₆ alkylene-R⁵, —C₁₋₆ alkylene-L-R⁵ or —C₁₋₆ alkylene-L-C₁₋₆ alkylene-R⁵ and L is not —C(O)—; and when R² is -L-R⁵ or -L-C₁₋₆ alkylene-R⁵, then L is not —O—, —S—, —NHS(O)₂— or —NHC(O)—;
each R³ is independently hydrogen, deuterium, C₁₋₆ alkyl, cycloalkyl, aryl, heteroaryl or heterocyclyl;
   wherein said C₁₋₆ alkyl is optionally substituted with one, two or three substituents independently selected from the group consisting of halo, —NO₂, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;
      wherein said cycloalkyl, aryl, heterocyclyl or heteroaryl are optionally further substituted with one, two or three substituents independently selected from the group consisting of halo, —NO₂, C₁₋₆ alkyl, aralkyl, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and
         wherein said C₁₋₆ alkyl, aralkyl, cycloalkyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;
            or when Y is —C(O)—, then R² and one of R³ can join together with the atom to which they are attached to form a heterocyclyl;
wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and
   wherein said C₁₋₆ alkyl is optionally substituted with one, two or three substituents independently selected from the group consisting of halo and heteroaryl;
      each R⁴ is independently hydrogen, deuterium, C₁₋₆ alkyl, —C(O)—OR²⁶, —C(O)—N(R²⁶)(R²⁶), cycloalkyl, aryl, heteroaryl or heterocyclyl;
wherein said C₁₋₆ alkyl is optionally substituted with one, two or three substituents independently selected from the group consisting of halo, —NO₂, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;
   wherein said cycloalkyl, aryl, heterocyclyl or heteroaryl are optionally further substituted with one, two or three substituents independently selected from the group consisting of halo, —NO₂, C₁₋₆ alkyl, aralkyl, cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and
      wherein said C₁₋₆ alkyl, aralkyl, cycloalkyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;
         or two R³ or two R⁴ together with the carbon atom to which they are attached form an oxo;
         R⁵ is cycloalkyl, aryl, heteroaryl or heterocyclyl;
wherein said cycloalkyl, aryl, heteroaryl or heterocyclyl are optionally substituted with one, two or three substituents independently selected from the group consisting of C₁₋₆ alkyl, C₂₋₄ alkynyl, halo, —NO₂, cycloalkyl, aryl, heterocyclyl, heteroaryl, —N(R²⁰)(R²²), —N(R²⁰) S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰;
   wherein said C₁₋₆ alkyl, cycloalkyl, aryl, heterocyclyl or heteroaryl are optionally further substituted with one, two or three substituents independently selected from the group consisting of halo, —NO₂, C₁₋₆ alkyl, cycloalkyl, aryl, heterocyclyl, heteroaryl, —N(R²⁰)(R²²), R²⁰, C(O)—OR²⁰, —C(O)—N(R²⁰)(R²²), —CN and —O—R²⁰; and
      wherein said C₁₋₆ alkyl, cycloalkyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;
         R⁶ is hydrogen, C₁₋₆ alkyl or cycloalkyl;
wherein said C₁₋₆ alkyl is optionally substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;
   R⁷ is hydrogen, halo, —O—R²⁰ or C₁₋₆ alkyl;
   R¹¹ is hydrogen or C₁₋₄ alkyl;
   R²⁰ and R²² are in each instance independently selected from the group consisting of hydrogen, C₁₋₆ alkyl, C₂₋₆ alkenyl, C₂₋₆ alkynyl, cycloalkyl, heterocyclyl, aryl and heteroaryl;
wherein the C₁₋₆ alkyl, C₂₋₆ alkenyl, C₂₋₆ alkynyl, cycloalkyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, cycloalkyl and heteroaryl; and
   wherein said heteroaryl is optionally further substituted with C₁₋₄ alkyl or cycloalkyl; or
      when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃, —OCF₃, aryl, heteroaryl and cycloalkyl; and
      each R²⁶ is independently selected from the group consisting of hydrogen, C₁₋₄ alkyl, aryl and cycloalkyl;
wherein the C₁₋₄ alkyl, aryl and cycloalkyl may be further substituted with from 1 to 3 substituents independently selected from the group consisting of hydroxyl, halo, C₁₋₄ alkoxy, —CF₃ and —OCF₃;
   or a pharmaceutically acceptable salt, ester, stereoisomer, mixture of stereoisomers or tautomer thereof;
   provided that when Y is —C(O)—, X is —O—, each R⁴ is hydrogen, R² and R³ together with the atom to which they are attached form a piperazine which is optionally substituted with tert-butoxycarbonyl and Q is a bond, then R¹ is not unsubstituted phenyl or morpholinyl; and that when Y is S(O)₂—, X is —O—, R² is benzyl, each R³ is hydrogen, Z⁴ is C-Q-R¹, Q is a bond and R¹ is aryl or heteroaryl, then both R⁴ are hydrogen.

In another embodiment, the present disclosure relates to a method of treating atrial fibrillation, atrial fluter or heart failure comprising administering a CAMK inhibitor (e.g AIP, KN-93) in combination with a late I_{Na} inhibitor according to the compound of Formula I selected from the group consisting of:
4-((3-methyloxetan-3-yl)methyl)-7-(4-(trifluoromethoxy)phenyl)-3,4-dihydrobenzo[f][1,4]oxazepin-5(2H)-one (II-1);
4-(2-(pyrrolidin-1-yl)ethyl)-7-(4-(trifluoromethoxy)phenyl)-3,4-dihydrobenzo[f][1,4]oxazepin-5(2H)-one (II-3);
4-((5-cyclobutyl-1,3,4-oxadiazol-2-yl)methyl)-7-(4-(trifluoromethoxy)phenyl)-3,4-dihydrobenzo[f][1,4]oxazepin-5(2H)-one (II-4);
4-((2,3-dihydrobenzo[b][1,4]dioxin-6-yl)methyl)-7-(4-(trifluoromethoxy)phenyl)-3,4-dihydrobenzo[f][1,4]oxazepin-5(2H)-one (II-5);
4-(quinolin-2-ylmethyl)-7-(4-(trifluoromethoxy)phenyl)-3,4-dihydrobenzo[f][1,4]oxazepin-5(2H)-one (II-7);
(R)-2-(pyrimidin-2-ylmethyl)-8-(4-(trifluoromethyl)phenyl)-3,4,12,12a-tetrahydro-1H-benzo[f]pyrazino[2,1-c][1,4]oxazepin-6(2H)-one (II-8);
4-(cyclopropylmethyl)-7-(4-(trifluoromethoxy)phenyl)-3,4-dihydrobenzo[f][1,4]oxazepin-5(2H)-one (II-10);
(S)-3-methyl-4-(pyrimidin-2-ylmethyl)-7-(4-(trifluoromethyl)phenyl)-3,4-dihydrobenzo[f][1,4]oxazepin-5(2H)-one (II-12);
(R)-3-methyl-4-(pyrimidin-2-ylmethyl)-7-(4-(trifluoromethyl)phenyl)-3,4-dihydrobenzo[f][1,4]oxazepin-5(2H)-one (II-13);
6-((5-oxo-7-(4-(trifluoromethoxy)phenyl)-2,3-dihydrobenzo[f][1,4]oxazepin-4(5H)-yl)methyl)picolinonitrile (II-14);
7-(4-(trifluoromethoxy)phenyl)-4-((6-(trifluoromethyl)pyridin-2-yl)methyl)-3,4-dihydrobenzo[f][1,4]oxazepin-5(2H)-one (II-15);
7-(4-(trifluoromethoxy)phenyl)-4-((6-(trifluoromethyl)pyridin-3-yl)methyl)-3,4-dihydrobenzo[f][1,4]oxazepin-5(2H)-one (II-16);
4-((6-methylpyridin-2-yl)methyl)-7-(4-(trifluoromethoxy)phenyl)-3,4-dihydrobenzo[f][1,4]oxazepin-5(2H)-one (II-17);
(2R,11aS)-2-amino-7-(4-(trifluoromethyl)phenyl)-2,3,11,11a-tetrahydrobenzo[f]pyrrolo[2,1-c][1,4]oxazepin-5(1H)-one (II-21);
(R)-2-(2,2-difluoroethyl)-8-(4-(trifluoromethyl)phenyl)-3,4,12,12a-tetrahydro-1H-benzo[f]pyrazino[2,1-c][1,4]oxazepin-6(2H)-one (II-22);
(R)-2-ethyl-8-(4-(trifluoromethyl)phenyl)-3,4,12,12a-tetrahydro-1H-benzo[f]pyrazino[2,1-c][1,4]oxazepin-6(2H)-one (II-23);
(S)-2-(2,2-difluoroethyl)-8-(4-(trifluoromethyl)phenyl)-3,4,12,12a-tetrahydro-1H-benzo[f]pyrazino[2,1-c][1,4]oxazepin-6(2H)-one (II-24);
(S)-2-ethyl-8-(4-(trifluoromethyl)phenyl)-3,4,12,12a-tetrahydro-1H-benzo[f]pyrazino[2,1-c][1,4]oxazepin-6(2H)-one (II-25);
4-(pyrazin-2-ylmethyl)-7-(4-(trifluoromethoxy)phenyl)-3,4-dihydrobenzo[f][1,4]oxazepin-5(2H)-one (II-31);
4-((5-methyloxazol-2-yl)methyl)-7-(4-(trifluoromethoxy)phenyl)-3,4-dihydrobenzo[f][1,4]oxazepin-5(2H)-one (II-33);
7-(4-(trifluoromethoxy)phenyl)-4-(2-(2,5,5-trimethyl-1,3-dioxan-2-yl)ethyl)-3,4-dihydrobenzo[f][1,4]oxazepin-5(2H)-one (II-35);
tert-butyl (2R,11aR)-5-oxo-7-(4-(trifluoromethyl)phenyl)-1,2,3,5,11,11a-hexahydrobenzo[f]pyrrolo[2,1-c][1,4]oxazepin-2-ylcarbamate (II-39);
4((5-(pyridin-2-yl)isoxazol-3-yl)methyl)-7-(4-(trifluoromethoxy)phenyl)-3,4-dihydrobenzo[f][1,4]oxazepin-5(2H)-one (II-41);
4-((4,6-dimethoxypyrimidin-2-yl)methyl)-7-(4-(trifluoromethoxy)phenyl)-3,4-dihydrobenzo[f][1,4]oxazepin-5(2H)-one (II-42);
ethyl 3-((5-oxo-7-(4-(trifluoromethoxy)phenyl)-2,3-dihydrobenzo[f][1,4]oxazepin-4(5H)-yl)methyl)benzoate (II-43);
4-(2-(pyrimidin-2-yl)ethyl)-7-(4-(trifluoromethoxy)phenyl)-3,4-dihydrobenzo[f][1,4]oxazepin-5(2H)-one (II-44);
4-(3,4-difluorobenzyl)-7-(4-(trifluoromethoxy)phenyl)-3,4-dihydrobenzo[f][1,4]oxazepin-5(2H)-one (II-45);
4-(2-chlorobenzyl)-7-(4-(trifluoromethoxy)phenyl)-3,4-dihydrobenzo[f][1,4]oxazepin-5(2H)-one (II-47);
4-(2,6-dichlorobenzyl)-7-(4-(trifluoromethoxy)phenyl)-3,4-dihydrobenzo[f][1,4]oxazepin-5(2H)-one (II-48);
4-(2,6-difluorobenzyl)-7-(4-(trifluoromethoxy)phenyl)-3,4-dihydrobenzo[f][1,4]oxazepin-5(2H)-one (II-49);
4-(2-(1H-pyrazol-1-yl)-ethyl)-7-(4-(trifluoromethoxy)phenyl)-3,4-dihydrobenzo[f][1,4]oxazepin-5(2H)-one (II-50);
(2S,11aS)-2-amino-7-(4-(trifluoromethyl)phenyl)-2,3,11,11a-tetrahydrobenzo[f]pyrrolo[2,1-c][1,4]oxazepin-5(1H)-one (II-51);
4-(2-(pyridin-2-yl)ethyl)-7-(4-(trifluoromethoxy)phenyl)-3,4-dihydrobenzo[f][1,4]oxazepin-5(2H)-one (II-54);
4-(2-fluorobenzyl)-7-(4-(trifluoromethoxy)phenyl)-3,4-dihydrobenzo[f][1,4]oxazepin-5(2H)-one (II-57);
(R)-7-(4-(trifluoromethyl)phenyl)-2,3,11,11a-tetrahydrobenzo[f]pyrrolo[2,1-c][1,4]oxazepin-5(1H)-one (II-59);
4-(pyrimidin-2-ylmethyl)-7-(4-(trifluoromethyl)phenyl)-3,4-dihydrobenzo[f][1,4]oxazepin-5(2H)-one (II-61);
4-(4-fluorobenzyl)-7-(4-(trifluoromethoxy)phenyl)-3,4-dihydrobenzo[f][1,4]oxazepin-5(2H)-one (II-62);
4-((1-methyl-1H-pyrazol-3-yl)methyl)-7-(4-(trifluoromethoxy)phenyl)-3,4-dihydrobenzo[f][1,4]oxazepin-5(2H)-one (II-64);
4-((5-chloropyrimidin-2-yl)methyl)-7-(4-(trifluoromethoxy)phenyl)-3,4-dihydrobenzo[f][1,4]oxazepin-5(2H)-one (II-65);
4-(pyridin-4-ylmethyl)-7-(4-(trifluoromethoxy)phenyl)-3,4-dihydrobenzo[f][1,4]oxazepin-5(2H)-one (II-67);
4-(5-cyclopropyl-1,3,4-oxadiazol-2-yl)methyl)-7-(4-(trifluoromethoxy)phenyl)-3,4-dihydrobenzo[f][1,4]oxazepin-5(2H)-one (II-68);
4-(2-(pyrimidin-2-yloxy)ethyl)-7-(4-(trifluoromethoxy)phenyl)-3,4-dihydrobenzo[f][1,4]oxazepin-5(2H)-one (II-69);
4-(pyridin-3-ylmethyl)-7-(4-(trifluoromethoxy)phenyl)-3,4-dihydrobenzo[f][1,4]oxazepin-5(2H)-one (II-70);
4-(pyridin-2-ylmethyl)-7-(4-(trifluoromethoxy)phenyl)-3,4-dihydrobenzo[f][1,4]oxazepin-5(2H)-one (II-72);
4-(pyrimidin-2-ylmethyl)-7-(4-(trifluoromethoxy)phenyl)-3,4-dihydrobenzo[f][1,4]oxazepin-5(2H)-one (II-73);
4-((3-methylpyridin-2-yl)methyl)-7-(4-(trifluoromethoxy)phenyl)-3,4-dihydrobenzo[f][1,4]oxazepin-5(2H)-one (II-75);
(R)-2-(2,2,2-trifluoroethyl)-8-(4-(trifluoromethyl)phenyl)-3,4,12,12a-tetrahydro-1H-benzo[f]pyrazino[2,1-e][1,4]oxazepin-6(2H)-one (II-83);
4-(pyrimidin-2-ylmethyl)-7-p-tolyl-3,4-dihydrobenzo[f][1,4]oxazepin-5(2H)-one (II-87);
7-(4-chlorophenyl)-4-(pyrimidin-2-ylmethyl)-3,4-dihydrobenzo[f][1,4]oxazepin-5(2H)-one (II-88);
7-(4-isopropylphenyl)-4-(pyrimidin-2-ylmethyl)-3,4-dihydrobenzo[f][1,4]oxazepin-5(2H)-one (II-89);
7-(4-ethylphenyl)-4-(pyrimidin-2-ylmethyl)-3,4-dihydrobenzo[f][1,4]oxazepin-5(2H)-one (II-91);
7-(4-cyclopropylphenyl)-4-(pyrimidin-2-ylmethyl)-3,4-dihydrobenzo[f][1,4]oxazepin-5(2H)-one (II-92);
(R)-4-(1-(pyrimidin-2-yl)ethyl)-7-(4-(trifluoromethyl)phenyl)-3,4-dihydrobenzo[f][1,4]oxazepin-5(2H)-one (II-95);
7-(4-isobutoxyphenyl)-4-(pyrimidin-2-ylmethyl)-3,4-dihydrobenzo[f][1,4]oxazepin-5(2H)-one (II-97);
7-(4-tert-butylphenyl)-4-(pyrimidin-2-ylmethyl)-3,4-dihydrobenzo[f][1,4]oxazepin-5(2H)-one (II-98);
7-(4-cyclopropoxyphenyl)-4-(pyrimidin-2-ylmethyl)-3,4-dihydrobenzo[f][1,4]oxazepin-5(2H)-one (II-102);
7-(4-fluorophenyl)-4-(pyrimidin-2-ylmethyl)-3,4-dihydrobenzo[f][1,4]oxazepin-5(2H)-one (II-104);
7-(2-fluoro-4-(trifluoromethyl)phenyl)-4-(pyrimidin-2-ylmethyl)-3,4-dihydrobenzo[f][1,4]oxazepin-5(2H)-one (II-105);
7-(3-fluoro-4-(2,2,2-trifluoroethoxy)phenyl)-4-(pyrimidin-2-ylmethyl)-3,4-dihydrobenzo[f][1,4]oxazepin-5(2H)-one (II-106);
4-(pyrimidin-2-ylmethyl)-7-(4-(2,2,2-trifluoroethoxy)phenyl)-3,4-dihydrobenzo[f][1,4]oxazepin-5(2H)-one (II-107);
7-(2-chloro-4-(trifluoromethyl)phenyl)-4-(pyrimidin-2-ylmethyl)-3,4-dihydrobenzo[f][1,4]oxazepin-5(2H)-one (II-110);
7-(4-(trifluoromethoxy)phenyl)-4-((4-(trifluoromethyl)pyrimidin-2-yl)methyl)-3,4-dihydrobenzo[f][1,4]oxazepin-5(2H)-one (II-113);
7-(4-(trifluoromethoxy)phenyl)-4-((5-(6-(trifluoromethyl)pyridin-3-yl)pyrimidin-2-yl)methyl)-3,4-dihydrobenzo[f][1,4]oxazepin-5(2H)-one (II-115);
7-(4-chloro-2-fluorophenyl)-4-(pyrimidin-2-ylmethyl)-3,4-dihydrobenzo[f][1,4]oxazepin-5(2H)-one (II-117);
1-(4-(5-oxo-4-(pyrimidin-2-ylmethyl)-2,3,4,5-tetrahydrobenzo[f][1,4]oxazepin-7-yl)phenyl)cyclopentanecarbonitrile (II-122);
7-(4-ethoxyphenyl)-4-(pyrimidin-2-ylmethyl)-3,4-dihydrobenzo[f][1,4]oxazepin-5(2H)-one (II-123);
7-(4-(difluoromethyl)phenyl)-4-(pyrimidin-2-ylmethyl)-3,4-dihydrobenzo[f][1,4]oxazepin-5(2H)-one (II-124);
4-(imidazo[1,2-a]pyridin-2-ylmethyl)-7-(4-(trifluoromethyl)phenyl)-3,4-dihydrobenzo[f][1,4]oxazepin-5(2H)-one (II-129);

4-((4(4-morpholinopyrimidin-2-yl)methyl)-7-(4-(trifluoromethoxy)phenyl)-3,4-dihydrobenzo[f][1,4]oxazepin-5(2H)-one (II-133);
4-benzyl-7-(4-(trifluoromethoxy)phenyl)-3,4-dihydrobenzo[f][1,4]oxazepin-5(2H)-one (II-134);
4-(imidazo[1,2-a]pyridin-2-ylmethyl)-7-(4-(trifluoromethyl)phenyl)-3,4-dihydrobenzo[f][1,4]oxazepin-5(2H)-one (II-135);
7-(3-fluoro-4-(trifluoromethyl)phenyl)-4-(pyrimidin-2-ylmethyl)-3,4-dihydrobenzo[f][1,4]oxazepin-5(2H)-one (II-136);
4-((4-methoxypyrimidin-2-yl)methyl)-7-(4-(trifluoromethoxy)phenyl)-3,4-dihydrobenzo[f][1,4]oxazepin-5(2H)-one (II-137);
4-((4-methylpyrimidin-2-yl)methyl)-7-(4-(trifluoromethoxy)phenyl)-3,4-dihydrobenzo[f][1,4]oxazepin-5(2H)-one (II-138);
4-((4-(piperidin-1-yl)pyrimidin-2-yl)methyl)-7-(4-(trifluoromethoxy)phenyl)-3,4-dihydrobenzo[f][1,4]oxazepin-5(2H)-one (II-139);
4-((4-(dimethylamino)pyrimidin-2-yl)methyl)-7-(4-(trifluoromethoxy)phenyl)-3,4-dihydrobenzo[f][1,4]oxazepin-5(2H)-one (II-140);
4-benzyl-7-(4-(trifluoromethyl)phenyl)-3,4-dihydrobenzo[f][1,4]oxazepin-5 (2H)-one
(II-141);
4-((3-methoxypyridin-2-yl)methyl)-7-(4-(trifluoromethoxy)phenyl)-3,4-dihydrobenzo[f][1,4]oxazepin-5(2H)-one (II-143);
7-(4-(cyclobutylmethoxy)phenyl)-4-(pyrimidin-2-ylmethyl)-3,4-dihydrobenzo[f][1,4]oxazepin-5(2H)-one (II-144);
7-(2-methyl-4-(trifluoromethyl)phenyl)-4-(pyrimidin-2-ylmethyl)-3,4-dihydrobenzo[f][1,4]oxazepin-5(2H)-one (II-145);
7-(2-methyl-4-(trifluoromethoxy)phenyl)-4-(pyrimidin-2-ylmethyl)-3,4-dihydrobenzo[f][1,4]oxazepin-5(2H)-one (II-146);
4-((1-(difluoromethyl)-1H-pyrazol-3-yl)methyl)-7-(4-(trifluoromethoxy)phenyl)-3,4-dihydrobenzo[f][1,4]oxazepin-5(2H)-one (II-147);
7-(4-(trifluoromethoxy)phenyl)-4((3-(trifluoromethyl)-1H-pyrazol-1-yl)methyl)-3,4-dihydrobenzo[f][1,4]oxazepin-5(2H)-one (II-148);
4-(pyrimidin-2-ylmethyl)-7-(4-(2,2,2-trifluoroethyl)phenyl)-3,4-dihydrobenzo[f][1,4]oxazepin-5(2H)-one (II-150);
4-(pyridin-2-ylmethyl)-7-(4-(2,2,2-trifluoroethyl)phenyl)-3,4-dihydrobenzo[f][1,4]oxazepin-5(2H)-one (II-151);
4((1-cyclopentyl-1H-pyrazol-3-yl)methyl)-7-(4-(trifluoromethoxy)phenyl)-3,4-dihydrobenzo[f][1,4]oxazepin-5(2H)-one (II-152);
4-((1-ethyl-1H-pyrazol-3-yl)methyl)-7-(4-(trifluoromethoxy)phenyl)-3,4-dihydrobenzo[f][1,4]oxazepin-5(2H)-one (II-153);
4-((1-methyl-1H-imidazol-4-yl)methyl)-7-(4-(trifluoromethoxy)phenyl)-3,4-dihydrobenzo[f][1,4]oxazepin-5(2H)-one (II-154);
4((4-methyl-1H-pyrazol-1-yl)methyl)-7-(4-(trifluoromethoxy)phenyl)-3,4-dihydrobenzo[f][1,4]oxazepin-5(2H)-one (II-155);
4-((4-chloro-1H-pyrazol-1-yl)methyl)-7-(4-(trifluoromethoxy)phenyl)-3,4-dihydrobenzo[f][1,4]oxazepin-5(2H)-one (II-156);
7-(4-(difluoromethyl)phenyl)-4-(pyridin-2-ylmethyl)-3,4-dihydrobenzo[f][1,4]oxazepin-5(2H)-one (II-157);
7-(4-chloro-3-fluorophenyl)-4-(pyridin-2-ylmethyl)-3,4-dihydrobenzo[f][1,4]oxazepin-5(2H)-one (II-158);
7-(4-(difluoromethoxy)phenyl)-4-(pyridin-2-ylmethyl)-3,4-dihydrobenzo[f][1,4]oxazepin-5(2H)-one (II-159);
4-((1-methyl-1H-pyrazol-4-yl)methyl)-7-(4-(trifluoromethoxy)phenyl)-3,4-dihydrobenzo[f][1,4]oxazepin-5(2H)-one (II-160);
4-(pyrimidin-2-ylmethyl)-7-(2,3,4-trifluorophenyl)-3,4-dihydrobenzo[f][1,4]oxazepin-5(2H)-one (II-162);
7-(3,4-difluorophenyl)-4-(pyrimidin-2-ylmethyl)-3,4-dihydrobenzo[f][1,4]oxazepin-5(2H)-one (II-163);
4-((3-fluoropyridin-2-yl)methyl)-7-(4-(trifluoromethoxy)phenyl)-3,4-dihydrobenzo[f][1,4]oxazepin-5(2H)-one (II-164);
4-benzyl-9-fluoro-7-(4-(trifluoromethoxy)phenyl)-3,4-dihydrobenzo[f][1,4]oxazepin-5(2H)-one (II-165);
4-benzyl-9-fluoro-7-(4-(trifluoromethyl)phenyl)-3,4-dihydrobenzo[f][1,4]oxazepin-5(2H)-one (II-166);
4-benzyl-8-fluoro-7-(4-(trifluoromethoxy)phenyl)-3,4-dihydrobenzo[f][1,4]oxazepin-5(2H)-one (II-167);
4-benzyl-8-fluoro-7-(4-(trifluoromethyl)phenyl)-3,4-dihydrobenzo[f][1,4]oxazepin-5(2H)-one (II-168);
7-(4-chloro-3-fluorophenyl)-4-((3-fluoropyridin-2-yl)methyl)-3,4-dihydrobenzo[f][1,4]oxazepin-5(2H)-one (II-169);
7-(2-fluoro-4-(trifluoromethyl)phenyl)-4((3-fluoropyridin-2-yl)methyl)-3,4-dihydrobenzo[f][1,4]oxazepin-5(2H)-one (II-170);
4-(5-oxo-4-(pyrimidin-2-ylmethyl)-2,3,4,5-tetrahydrobenzo[f][1,4]oxazepin-7-yl)phenyl trifluoromethanesulfonate (II-171);
4-((5-methylpyrazin-2-yl)methyl)-7-(4-(trifluoromethoxy)phenyl)-3,4-dihydrobenzo[f][1,4]oxazepin-5(2H)-one (II-172);
2,2,3,3-tetradeutero-4-(pyrimidin-2-ylmethyl)-7-(4-(trifluoromethoxy)phenyl)-3,4-dihydrobenzo[f][1,4]oxazepin-5(2H)-one (II-174);
4-((6-methylpyrazin-2-yl)methyl)-7-(4-(trifluoromethoxy)phenyl)-3,4-dihydrobenzo[f][1,4]oxazepin-5(2H)-one (II-175);
4-((3-fluoropyridin-2-yl)methyl)-7-(4-(trifluoromethyl)phenyl)-3,4-dihydrobenzo[f][1,4]oxazepin-5(2H)-one (II-176);
N-(2-(5-oxo-7-(4-(trifluoromethoxy)phenyl)-2,3-dihydrobenzo[f][1,4]oxazepin-4(5H)-yl)ethyl)benzenesulfonamide (II-177);
N-(2-(5-oxo-7-(4-(trifluoromethoxy)phenyl)-2,3-dihydrobenzo[f][1,4]oxazepin-4(5H)-yl)ethyl)cyclopropanesulfonamide (II-179);
4-((1-methyl-1H-imidazol-2-yl)methyl)-7-(4-(trifluoromethoxy)phenyl)-3,4-dihydrobenzo[f][1,4]oxazepin-5(2H)-one (II-186);
4-((1-benzyl-1H-imidazol-2-yl)methyl)-7-(4-(trifluoromethoxy)phenyl)-3,4-dihydrobenzo[f][1,4]oxazepin-5(2H)-one (II-187);
4-(imidazo[1,2-a]pyridin-2-ylmethyl)-7-(4-(trifluoromethoxy)phenyl)-3,4-dihydrobenzo[f][1,4]oxazepin-5(2H)-one (II-189);
N-cyclopropyl-3-(5-oxo-7-(4-(trifluoromethoxy)phenyl)-2,3-dihydrobenzo[f][1,4]oxazepin-4(5H)-yl)propane-1-sulfonamide (II-190);
N-(2-(5-oxo-7-(4-(trifluoromethoxy)phenyl)-2,3-dihydrobenzo[f][1,4]oxazepin-4(5H)-yl)ethyl)pyrimidine-2-carboxamide (II-192);
7-(4-(4-fluorophenoxy)phenyl)-4-(pyrimidin-2-ylmethyl)-3,4-dihydrobenzo[f][1,4]oxazepin-5(2H)-one (II-193);
7-(4-phenoxyphenyl)-4-(pyrimidin-2-ylmethyl)-3,4-dihydrobenzo[f][1,4]oxazepin-5(2H)-one (II-194); and
7-(3-phenoxyphenyl)-4-(pyrimidin-2-ylmethyl)-3,4-dihydrobenzo[f][1,4]oxazepin-5(2H)-one (II-195);
   or a pharmaceutically acceptable salt, ester, stereoisomer, mixture of stereoisomers or tautomer thereof.

In yet another embodiment, the present disclosure provides a method of treating atrial fibrillation or heart failure comprising co-administration of a CAM Kinase inhibitor and a compound of Formula I selected from the group consisting of:
4-(pyrimidin-2-ylmethyl)-7-(4-(trifluoromethyl)phenyl)-3,4-dihydrobenzo[f][1,4]oxazepin-5(2H)-one, having the structure:

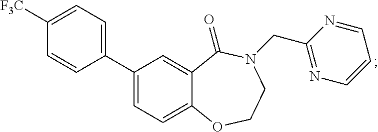 [SMILES: O=C1c2cc(-c3ccc(C(F)(F)F)cc3)ccc2OCCN1Cc1ncccn1]

4-(pyrimidin-2-ylmethyl)-7-(44 trifluoromethoxy)phenyl)-3,4-dihydrobenzo[f][1,4]oxazepin-5(2H)-one, having the structure:

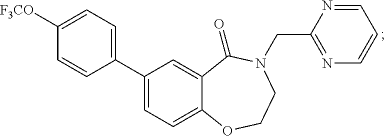 [SMILES: O=C1c2cc(-c3ccc(OC(F)(F)F)cc3)ccc2OCCN1Cc1ncccn1]

7-(4-chlorophenyl)-4-(pyrimidin-2-ylmethyl)-3,4-dihydrobenzo[f][1,4]oxazepin-5(2H)-one, having the structure:

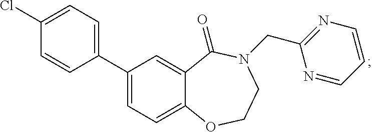 [SMILES: O=C1c2cc(-c3ccc(Cl)cc3)ccc2OCCN1Cc1ncccn1]

7-(4-tert-butylphenyl)-4-(pyrimidin-2-ylmethyl)-3,4-dihydrobenzo HI [1,4]oxazepin-5(2H)-one, having the structure:

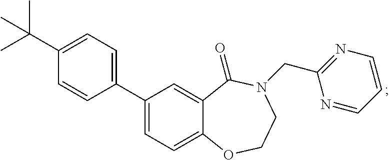 [SMILES: CC(C)(C)c1ccc(-c2ccc3c(c2)C(=O)N(Cc2ncccn2)CCO3)cc1]

4-(imidazo[1,2-a]pyridin-2-ylmethyl)-7-(4-(trifluoromethyl)phenyl)-3,4-dihydrobenzo[f][1,4]oxazepin-5(2H)-one, having the structure:

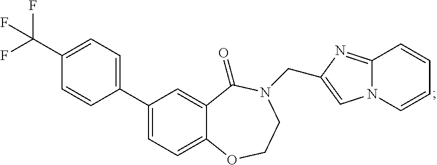 [SMILES: O=C1c2cc(-c3ccc(C(F)(F)F)cc3)ccc2OCCN1Cc1cn2ccccc2n1]

or a pharmaceutically acceptable salt thereof.
Compounds of Formula I are disclosed in U.S. Pat. No. 8,697,863, the entirety of which is incorporated herein by reference.

In another embodiment, the present disclosure provides a method for treating atrial fibrillation, atrial flutter or heart failure comprising co-administering a therapeutically effective amount of a CAMK inhibitor (e.g. KN-93 or AIP) and a compound of formula II:

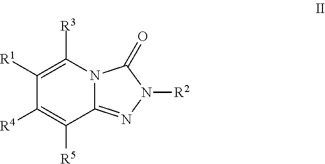

wherein:
R¹ is aryl or heteroaryl,
wherein said aryl or heteroaryl are optionally substituted with one, two, or three substituents independently selected from the group consisting of hydroxyl, halo, —NO₂, CN, —SF₅, —Si(CH₃)₃—O—CF₃, —O—R²⁰, —S—R²⁰, —C(O)—R²⁰, C(O)OH, —N(R²⁰)(R²²), —C(O)—N(R²⁰)(R²²), —N(R²⁰)—C(O)—R²², —N(R²⁰)—S(═O)₂—R²⁶, —S(═O)₂—R²⁰, —S(═O)₂—N(R²⁰)(R²²), C₁-C₃ alkoxy, C₁₋₄ alkyl, C₂₋₄ alkenyl, C₂₋₄ alkynyl, cycloalkyl, heteroaryl, and heterocyclyl;
   wherein said alkoxy, alkyl, alkenyl, alkynyl, heteroaryl, cycloalkyl, or heterocyclyl are optionally substituted with one, two, or three substituents independently selected from hydroxyl, halo, —NO₂, —O—CF₃, —O—CF₂, phenyl, heterocyclyl, heteroaryl, cycloalkyl, —N(R²⁰)(R²²), —C(O)—R²⁰, —C(O)—O—R²⁰, —C(O)—N(R²⁰)(R²²), —CN, and —O—R²⁰,
R² is hydrogen, C₁₋₁₅ alkyl, C₁₋₄ alkoxy, —C(O)—O—R²⁶, —C(O)—N(R²⁶)(R²⁸), —N(R²⁰)—S(═O)₂—R²⁰, cycloalkyl, aryl, heteroaryl, heterocyclyl,
wherein said alkyl is optionally substituted with one, two, or three substituents independently selected from hydroxyl, alkoxy, halo, —NO₂, —O—CF₃, —O—CF₂, aryl, heterocyclyl, heteroaryl, cycloalkyl, —N(R²⁰)(R²²), —C(O)—R²⁰, —C(O)—O—R²⁰, —C(O)—N(R²⁰)(R²²), —CN, and O—R²⁰, and
   wherein said alkoxy, cycloalkyl, aryl, heterocyclyl, or heteroaryl are optionally further substituted with one, two, or three substituents independently selected from hydroxyl, halo, —NO₂, —O—CF₃, C₁₋₆ alkyl, C₁₋₄ alkoxy, benzyl, aryl, heterocyclyl, heteroaryl, cycloalkyl, —N(R²⁰)(R²²), —C(O)—R²⁰, —C(O)—O—R²⁰, —C(O)—N(R²⁰)(R²²), —CN, and —O—R²⁰; and
      wherein said C₁₋₆ alkyl, C₁₋₄ alkoxy, benzyl, aryl, heterocyclyl, heteroaryl, cycloalkyl, are optionally further substituted with one, two, or three substituents independently selected from hydroxyl, halo, —NO₂, —O—CF₃, —CF₃, —O—CHF₂, —N(R²⁰)(R²²), —C(O)—N(R²⁰), —C(O)—R²⁰, —C(O)—N(R²⁰)(R²²), —CN, and —O—R²⁰
R³ is selected from the group consisting of hydrogen, hydroxyl, halo, C₁₋₄ alkyl, C₁-C₃ alkoxy, —R²⁵—N(R²⁰)(R²²), —R²⁵—C(O)—O—R²⁰, —R²⁵—C(O)—N(R²⁰)(R²²), —R²⁵—C(O)—O—N(R²⁰)(R²²), —R²⁵—N(R²⁰)—C(O)—R²², and —R²⁵—O—C(O)—N(R²⁰)(R²²),
   wherein said alkyl are optionally substituted with one, two, or three substituents independently selected from hydroxyl, halo,
R⁴ is selected from the group consisting of hydrogen, optionally substituted alkyl, —CF₃, -halo, and —O—R²⁴;
R⁵ is selected from the group consisting of hydrogen, optionally substituted alkyl, amino, optionally substituted alkoxy, —CF₃, —O—CF₃, —CN, and —N(R²⁰)C(O)—R²²;
R²⁰ and R²² are in each instance independently selected from the group consisting of hydrogen, C₁-C₁₅ alkyl, C₂-C₁₅ alkenyl, C₂-C₁₅ alkynyl, cycloalkyl, heterocyclyl, aryl, and heteroaryl,
   wherein the alkyl, alkenyl, alkynyl, heterocyclyl, aryl, and heteroaryl are optionally substituted with one, two, or three substituents independently selected from hydroxyl, halo, C₁₋₄ alkyl, mono- or dialkylamino, alkyl or aryl or heteroaryl amide, —NO₂, —SO₂R²⁶, —CN, C₁₋₃ alkoxy, —CF₃, —OCF₃, aryl, cycloalkyl, and heteroaryl; or;
when R²⁰ and R²² are attached to a common nitrogen atom R²⁰ and R²² may join to form a heterocyclic ring which is then optionally substituted with one, two, or three substituents independently selected from hydroxyl, halo, alkyl, benzyl, phenyl, phenoxy, benzyloxy, mono- or dialkylamino, alkyl or aryl or heteroaryl amide, —NO₂, —SO₂R²⁶, —CN, C₁₋₃ alkoxy, —CF₃, and —OCF₃, aryl, cycloalkyl;
R²⁵ is in each instance independently a covalent bond or selected from C₁-C₃ alkylene optionally substituted with one or two C₁-C₃ alkyl groups; and
R²⁶ and R²⁸ are in each instance independently selected from hydrogen, alkyl, or cycloalkyl, wherein the alkyl, phenyl and cycloalkyl may be further substituted with from 1 to 3 substituents independently selected from hydroxyl, halo, C₁₋₄ alkoxy, —CF₃, and —CF₃;
   or a pharmaceutically acceptable salt, ester, prodrug, or solvate thereof.

In another embodiment, the present disclosure relates to a method of treating atrial fibrillation, atrial flutter or heart failure comprising administering a CAMK inhibitor (e.g. AIP, KN-93) in combination with a late I_{Na} inhibitor according to the compound of Formula II selected from the group consisting of:
2-((3-methyl-1,2,4-oxadiazol-5-yl)methyl)-6-(4-(trifluoromethoxy)phenyl)-[1,2,4]triazolo[4,3-a]pyridin-3(2H)-one;
2-((4-methyl-1,2,5-oxadiazol-3-yl)methyl)-6-(4-(trifluoromethoxy)phenyl)-[1,2,4]triazolo[4,3-a]pyridin-3(2H)-one;
2-((5-methyl-1,2,4-oxadiazol-3-yl)methyl)-6-(4-(trifluoromethoxy)phenyl)-[1,2,4]triazolo[4,3-a]pyridin-3(2H)-one;
2-(2-hydroxy-3-(2-methoxyphenoxy)propyl)-6-(4-(trifluoromethoxy)phenyl)-[1,2,4]triazolo[4,3-a]pyridin-3(2H)-one;
2-((5-methylisoxazol-3-yl)methyl)-6-(4-(trifluoromethoxy)phenyl)[1,2,4]triazolo[4,3-a]pyridin-3 (2H)-one;
2-((3,5-dimethylisoxazol-4-yl)methyl)-6-(4-(trifluoromethoxy)phenyl)-[1,2,4]triazolo[4,3-a]pyridin-3(2H)-one;
6-(4-(trifluoromethoxy)phenyl)-2-((5-(3-(trifluoromethyl)phenyl)-1,2,4-oxadiazol-3-yl)methyl)-[1,2,4]triazolo[4,3-a]pyridin-3(2H)-one;
2-(3-(5-methyl-1,2,4-oxadiazol-3-yl)benzyl)-6-(4-(trifluoromethoxy)phenyl)-[1,2,4]triazolo[4,3-a]pyridin-3(2H)-one;
2-((3-(2,6-dichlorophenyl)-1,2,4-oxadiazol-5-yl)methyl)-6-(4-(trifluoromethoxy)phenyl)-[1,2,4]triazolo[4,3-a]pyridin-3(2H)-one;
2-(benzo[c][1,2,5]oxadiazol-5-ylmethyl)-6-(4-(trifluoromethoxy)phenyl)-[1,2,4]triazolo[4,3-a]pyridin-3 (2H)-one;
2-((5-(2-methoxyphenyl)-1,2,4-oxadiazol-3-yl)methyl)-6-(4-(trifluoromethoxy)phenyl)-[1,2,4]triazolo[4,3-a]pyridin-3(2H)-one;
2-(quinolin-2-ylmethyl)-6-(4-(trifluoromethoxy)phenyl)-[1,2,4]triazolo[4,3-a]pyridin-3 (2H)-one;
2-((4-phenyl-1,2,5-oxadiazol-3-yl)methyl)-6-(4-(trifluoromethoxy)phenyl)-[1,2,4]triazolo[4,3-a]pyridin-3(214)-one;
2-(oxazol-2-ylmethyl)-6-(4-(trifluoromethoxy)phenyl)-[1,2,4]triazolo[4,3-a]pyridin-3 (2H)-one;
2-(benzo[d]thiazol-2-ylmethyl)-6-(4-(trifluoromethoxy)phenyl)-[1,2,4]triazolo[4,3-a]pyridin-3(2H)-one;
2-((4,5-dimethyloxazol-2-yl)methyl)-6-(4-(trifluoromethoxy)phenyl)-[1,2,4]triazolo[4,3-a]pyridin-3 (2H)-one;
2-(2-methoxy-3-(2-methoxyphenoxy)propyl)-6-(4-(trifluoromethoxy)phenyl)-[1,2,4]triazolo[4,3-a]pyridin-3(2H)-one;
2-(3-phenoxypropyl)-6-(4-(trifluoromethoxy)phenyl)-[1,2,4]triazolo[4,3-a]pyridin-3 (2H)-one;
2-((5-methyloxazol-2-yl)methyl)-6-(4-(trifluoromethoxy)phenyl)-[1,2,4]triazolo[4,3-a]pyridin-3 (2H)-one;
2-((3-cyclopropyl-1-methyl-1H-pyrazol-5-yl)methyl)-6-(4-(trifluoromethoxy)phenyl)-[1,2,4]triazolo[4,3-a]pyridin-3 (2H)-one;
2-(2-phenoxyethyl)-6-(4-(trifluoromethoxy)phenyl)-[1,2,4]triazolo[4,3-a]pyridin-3 (2H)-one;
2-((3-benzyl-1,2,4-oxadiazol-5-yl)methyl)-6-(4-(trifluoromethoxy)phenyl)-[1,2,4]triazolo[4,3-a]pyridin-3 (2H)-one;
5-methoxy-2-((3-methyl-1,2,4-oxadiazol-5-yl)methyl)-6-(4-(trifluoromethoxy)phenyl)-[1,2,4]triazolo[4,3-a]pyridin-3 (2H)-one;
2-((5-methyl-1,2,4-oxadiazol-3-yl)methyl)-6-(3-phenoxyphenyl)-[1,2,4]triazolo[4,3-a]pyridin-3(2H)-one;
6-(4-(4-chlorophenoxy)phenyl)-2-((5-methyl-1,2,4-oxadiazol-3-yl)methyl)-[1,2,4]triazolo[4,3-a]pyridin-3 (2H)-one;
2-((3 (2-chlorophenyl)-1,2,4-oxadiazol-5-yl)methyl)-6-(4-(trifluoromethoxy)phenyl)-[1,2,4]triazolo[4,3-a]pyridin-3(2H)-one;
2-(pyridin-2-yl)-6(4-(trifluoromethoxy)phenyl)-[1,2,4]triazolo[4,3-a]pyridin-3 (2H)-one;
2-(2-(pyridin-2-yl)ethyl)-6-(4-(trifluoromethoxy)phenyl)-[1,2,4]triazolo[4,3-a]pyridin-3 (2H)-one;
2-(3-(2-methoxyphenoxy)propyl)-6-(4-(trifluoromethoxy)phenyl)-[1,2,4]triazolo[4,3-a]pyridin-3 (2H)-one;
2-(2-(3-phenoxypyrrolidin-1-yl)ethyl)-6-(4-(trifluoromethoxy)phenyl)-[1,2,4]triazolo[4,3-a]pyridin-3(2H)-one;
2-(2-(4-chlorophenoxy)ethyl)-6-(4-(trifluoromethoxy)phenyl)-[1,2,4]triazolo[4,3-a]pyridin-3 (2H)-one;
2-(2-(3-methyl-1H-pyrazol-1-yl)ethyl)-6-(4-(trifluoromethoxy)phenyl)-[1,2,4]triazolo[4,3-a]pyridin-3(2H)-one;
2-(2-hydroxy-3-phenoxypropyl)-6-(4-(trifluoromethoxy)phenyl)-[1,2,4]triazolo[4,3-a]pyridin-3 (2H)-one;
2-(2-(2,6-dimethylphenoxy)ethyl)-6-[4-(trifluoromethoxy)phenyl)-[1,2,4]triazolo[4,3-a]pyridin-3 (2H)-one;
2-(2-(2-chlorophenoxy)ethyl)-6-(4-(trifluoromethoxy)phenyl)-[1,2,4]triazolo[4,3-a]pyridin-3 (2H)-one;
6-(4-(trifluoromethoxy)phenyl)-2-(2-(3-(trifluoromethyl)pyridin-2-yloxy)ethyl)-[1,2,4]triazolo[4,3-a]pyridin-3 (2H)-one;
2-(2-(6-methylpyridin-2-yl)ethyl)-6-(4-(trifluoromethoxy)phenyl)-[1,2,4]triazolo[4,3-a]pyridin-3 (2H)-one;
2(2-(pyridin-2-yloxy)ethyl)-6-(4-(trifluoromethoxy)phenyl)-[1,2,4]triazolo[4,3-a]pyridin-3 (2H)-one;
2-(2-(4,4-difluoropiperidin-1-yl)ethyl)-6-(4-(trifluoromethoxy)phenyl)-[1,2,4]triazolo[4,3-a]pyridin-3 (2H)-one;
2-(3-(2-fluorophenoxy)propyl)-6-(4-(trifluoromethoxy)phenyl)-[1,2,4]triazolo[4,3-a]pyridin-3(2H)-one;
2-((3-(2-chlorophenoxy)propyl)-6-(4-(trifluoromethoxy)phenyl)-[1,2,4]triazolo[4,3-a]pyridin-3(2H)-one;
6-(4-(4-chlorophenoxy)phenyl)-2-((3-methyl-1,2,4-oxadiazol-5-yl)methyl)-[1,2,4]triazolo[4,3-a]pyridin-3(2H)-one;
2-(2-(3-chlorophenoxy)ethyl)-6-(4-(trifluoromethoxy)phenyl)[1,2,4]triazolo[4,3-a]pyridin-3 (2H)-one;
2-(2-(4-fluorophenoxy)ethyl)-6-(4-(trifluoromethoxy)phenyl)[1,2,4]triazolo[4,3-a]pyridin-3 (2H)-one;
2-(2-(3-bromopyridin-2-yl)ethyl)-6-(4-(trifluoromethoxy)phenyl)[1,2,4]triazolo[4,3-a]pyridin-3(2H)-one;
2-(4-fluorophenethyl)-6-(4-(trifluoromethoxy)phenyl)[1,2,4]triazolo[4,3-a]pyridin-3(2H)-one;
2-(2-(3-cyclopropylpyridin-2-yl)ethyl)-6-(4-(trifluoromethoxy)phenyl)-[1,2,4]triazolo[4,3-a]pyridin-3(2H)-one; and
2-(2-(3-methylpyridin-2-yl)ethyl)-6-(4-(trifluoromethoxy)phenyl)[1,2,4]triazolo[4,3-a]pyridin-3(2H)-one.

In yet another embodiment, the present disclosure provides a method of treating atrial fibrillation or heart failure comprising co-administering a CAM Kinase inhibitor and a compound of Formula II, which is 2-((3-methyl-1,2,4-oxadiazol-5-yl)methyl)-6-(4-(trifluoromethoxy)phenyl)[1,2,4]triazolo[4,3-a]pyridin-3(2H)-one; represented by the structure:

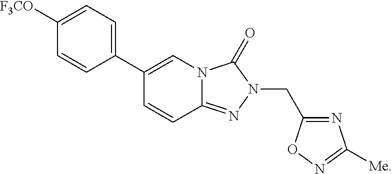 [SMILES: Cc1noc(Cn2nc3ccc(-c4ccc(OC(F)(F)F)cc4)cn3c2=O)n1]

Compounds of Formula II are disclosed in U.S. Patent publication US2012/0010192A1, published Jan. 12, 2012 and also disclosed in U.S. Pat. No. 8,703,759, the entireties of which are incorporated herein by reference.

In another embodiment, the present disclosure provides a method for treating atrial fibrillation, atrial flutter or heart failure comprising co-administering a therapeutically effective amount of a CAMK inhibitor (e.g. KN-93 or AIP) and a late I_{Na} compound of formula. III:

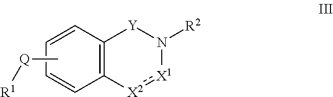

wherein:
the dotted line represents an optional double bond;
Y is —C(R⁵)₂— or —C(O)—;
X¹ is N and X² is N, X¹ is N and X² is CR³, or X¹ is CR³ and X² is N, and the dotted line is a double bond; or
X¹ is C(R³)₂ and X² is NR⁴, —O—, —S—, —S(O)— or —S(O)₂—, or X¹ and X² are both C(R³)₂, and the dotted line is a single bond;
   provided that:
   when the dotted line is a single bond and Y is —C(R⁵)₂—; then both X¹ and X² are C(R³)₂; and
   when the dotted line is a double bond; Y is —C(O)—;
Q is a covalent bond or C₂₋₄ alkynylene;
R¹ is C₃₋₆ cycloalkyl, C₃₋₆ cycloalkenyl, aryl, heterocyclyl or heteroaryl;
wherein said C₃₋₆ cycloalkyl, C₃₋₆ cycloalkenyl, aryl, heterocyclyl or heteroaryl are optionally substituted with one, two or three substituents independently selected from the group consisting of halo, —NO₂, CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—S(O)₂—R²², —S(O)₂—R²⁰, —S(O)₂—N(R²⁰)(R²²), C₁₋₄ alkyl, C₂₋₄ alkenyl, C₂₋₄ alkynyl, C₃₋₆ cycloalkyl, aryl, heteroaryl and heterocyclyl; and
   wherein said C₁₋₄ alkyl, C₂₋₄ alkenyl, C₂₋₄ alkynyl, C₃₋₆ cycloalkyl, aryl, heteroaryl or heterocyclyl are optionally substituted with one, two or three substituents independently selected from the group consisting of halo, —NO₂, aryl, heterocyclyl, heteroaryl, C₁₋₄ alkyl, C₃₋₆ cycloalkyl, —N(R²⁰)(R²²), —C(O)—R²⁰, —C(O)—OR²⁰, —C(O)—N(R²⁰)(R²²), —CN and —O—R²⁰;
R² is —R⁶, —C₁₋₆ alkylene-R⁶, —C₂₋₆ alkenylene-R⁶, —C₂₋₆ alkynylene-R⁶, -L-R⁶, -L-C₁₋₆ alkylene-R⁶, —C₁₋₆ alkylene-L-R⁶ or —C₁₋₆ alkylene-L-C₁₋₆ alkylene-R⁶;
L is —O—, —S—, —C(O)—, —S(O)₂—, —NR²⁰S(O)₂—, —S(O)₂NR²⁰—, —C(O)NR²⁰— or —NR²⁰C(O)—; provided that when Y is —C(R⁵)₂—, then L is C(O)— or —S(O)₂—, and R² is -L-R⁶, -L-C₁₋₆ alkylene-R⁶, alkylene-L-R⁶ or —C₁₋₆ alkylene-L-C₁₋₆ alkylene-R⁶;
each R³ is independently hydrogen, C₁₋₆ alkyl, C₃₋₆ cycloalkyl, aryl, heteroaryl or heterocyclyl;
   wherein said C₁₋₆ alkyl is optionally substituted with one, two or three substituents independently selected from the group consisting of halo, —NO₂, C₃₋₆ cycloalkyl, aryl, heterocyclyl, heteroaryl, —N(R²⁰)(R²²); —C(O)—R²⁰; —C(O)—OR²⁰, —C(O)—N(R²⁰)(R²²); —CN and —O—R²⁰;
      wherein said C₃₋₆ cycloalkyl, aryl, heterocyclyl and heteroaryl are optionally further substituted with one, two or three substituents independently selected from the group consisting of halo, —NO₂, C₁₋₆ alkyl, aralkyl, C₃₋₆ cycloalkyl, aryl, heterocyclyl, heteroaryl, —N(R²⁰)(R²²); —C(O)—R²⁰; —C(O)—OR²⁰, —C(O)—OR²⁰; —C(O)—N(R²⁰)(R²²); —CN and —O—R²⁰; and
         wherein said C₁₋₆ alkyl, aralkyl, C₃₋₆ cycloalkyl, aryl, heterocyclyl and heteroaryl are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;
or when X¹ is C(R³)₂, two R³ can join together with the with the carbon atom to which they are attached to form a C₃₋₆ cycloalkyl or heterocyclyl;
R⁴ is hydrogen, C₁₋₆ alkyl, C₁₋₄ alkoxy, —C(O)—OR²⁰, —C(O)—N(R²⁰)(R²²); —N(R²⁰)—S(O)₂—R²⁰, C₃₋₆ cycloalkyl, aryl, heteroaryl or heterocyclyl;
   wherein said C₁₋₆ alkyl is optionally substituted with one, two or three substituents independently selected from the group consisting of halo, —NO₂, C₃₋₆ cycloalkyl, aryl, heterocyclyl, heteroaryl, —N(R²⁰)(R²²), —C(O)R²⁰, —C(O)—OR²⁰, —C(O)—N(R²⁰)(R²²), —CN and —O—R²⁰;
   wherein said C₃₋₆ cycloalkyl, aryl, heterocyclyl or heteroaryl are optionally further substituted with one, two or three substituents independently selected from the group consisting of halo, —NO₂, C₁₋₆ alkyl, aralkyl, C₃₋₆ cycloalkyl, aryl, heterocyclyl, heteroaryl, —N(R²⁰)(R²²), —C(O)—R²⁰, —C(O)—OR²⁰, —C(O)—N(R²⁰)(R²²), —CN, and —O—R²⁰; and
      wherein said C₁₋₆ alkyl, aralkyl, C₃₋₆ cycloalkyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;
each R⁵ is independently hydrogen or C₁₋₆ alkyl;
R⁶ is C₃₋₆ cycloalkyl, aryl, heteroaryl or heterocyclyl;
   wherein said C₃₋₆ cycloalkyl, aryl, heteroaryl or heterocyclyl are optionally substituted with one, two or three substituents independently selected from the group consisting of C₁₋₆ alkyl, C₂₋₄ alkynyl, halo, —NO₂, C₃₋₆ cycloalkyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—N(R²⁰)(R²²), —S(O)₂—R²⁰, —CN and —O—R²⁰;
      wherein said C₁₋₆ alkyl, C₃₋₆ cycloalkyl, aryl, heterocyclyl or heteroaryl are optionally further substituted with one, two or three substituents independently selected from the group consisting of halo, —NO₂, C₁₋₆ alkyl, C₃₋₆ cycloalkyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and
         wherein said C₁₋₆ alkyl, C₃₋₆ cycloalkyl, aryl, heterocyclyl or heteroaryl are optionally further substituted with one, two or three substituents independently selected from the group consisting of C₁₋₆ alkyl, halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;
R²⁰ and R²² are in each instance independently hydrogen, C₁₋₆ alkyl, C₂₋₆ alkenyl, C₂₋₆ alkynyl, C₃₋₆ cycloalkyl, heterocyclyl, aryl or heteroaryl; and
   wherein the C₁₋₆ alkyl, C₂₋₆ alkenyl, C₂₋₆ alkynyl, C₃₋₆ cycloalkyl, heterocyclyl, aryl or heteroaryl are optionally substituted with one, two or three substituents independently selected from the group consisting of hydroxyl, halo, C₁₋₄ alkyl, aralkyl, —N(R²⁶)(R²⁸), aminoacyl, —NO₂, —S(O)₂—R²⁶, —CN, C₁₋₃ alkoxy, —CF₃, —OCF₃, —OCH₂CF₃, —C(O)—NH₂, —C(O)—R²⁶, —C(O)—OR²⁶, aryl, C₃₋₆ cycloalkyl, heterocyclyl, aryl and heteroaryl;
      wherein said aralkyl, heterocyclyl or heteroaryl is optionally further substituted with C₁₋₄ alkyl, —CF₃, aryl or C₃₋₆ cycloalkyl; or
when R²⁰ and R²² are attached to a common nitrogen atom R²⁰ and R²² may join to form a heterocyclic or heteroaryl ring which is then optionally substituted with one, two or three substituents independently selected from the group consisting of hydroxyl, halo, alkyl, aralkyl, aryl, aryloxy, aralkyloxy, heteroaryloxy, substituted amino, aminoacyl, —NO₂, —S(O)₂—R²⁶, —CN, C₁₋₃ alkoxy, hydroxymethyl, —CF₃, —OCF₃, aryl, heteroaryl and C₃₋₆ cycloalkyl; and
R²⁶ and R²⁸ are each independently selected from the group consisting of hydrogen, C₁₋₆ alkyl, C₁₋₆ alkenyl, C₃₋₆ cycloalkyl, aryl and heteroaryl; and
   wherein the C₁₋₆ alkyl, C₃₋₆ cycloalkyl, aryl or heteroaryl may be further substituted with from 1 to 3 substituents independently selected from the group consisting of hydroxyl, halo, C₁₋₄ alkoxy, —CF₃, —OCF₃ and C₃₋₆ cycloalkyl;

or a pharmaceutically acceptable salt, ester, stereoisomer, or tautomer thereof.

In another embodiment, the disclosure provides a method of treating atrial fibrillation or heart failure comprising co-administering a CAM Kinase inhibitor and a compound of Formula III selected from the group consisting of:
3-((3-methyl-1,2,4-oxadiazol-5-yl)methyl)-6-(4-(trifluoromethoxy)phenyl)benzo[d][1,2,3]triazin-4(3H)-one;
trifluoromethoxy)phenyl)benzo[d][1,2,3]triazin-4(3H)-one;
3-((5-3-((5-chloropyrimidin-2-yl)methyl)-6-(4-(methyl-1,2,4-oxadiazol-3-yl)methyl)-6-(4-(trifluoromethoxy)phenyl)benzo[d][1,2,3]triazin-4(3H)-one;
3-((3-methyl-1,2,4-oxadiazol-5-yl)methyl)-6-(4-phenoxyphenyl)benzo[d][1,2,3]triazin-4(3H)-one;
3-((3-phenylisoxazol-5-yl)methyl)-6-(4-(trifluoromethoxy)phenyl)benzo[d][1,2,3]triazin-4(3H)-one;
3-((3-benzyl-1,2,4-oxadiazol-5-yl)methyl)-6-(4-(trifluoromethoxy)phenyl)benzo[d][1,2,3]triazin-4(3H)-one;
3-(2-(1H-pyrazol-1-yl)ethyl)-6-(4-(trifluoromethoxy)phenyl)benzo[d][1,2,3]triazin-4(3H)-one;
3-((5-cyclopropyl-1,2,4-oxadiazol-3-yl)methyl)-6-(4-(trifluoromethoxy)phenyl)benzo[d][1,2,3]triazin-4(3H)-one;
3-(2-(pyridin-2-yl)ethyl)-6-(4-(trifluoromethoxy)phenyl)benzo[d][1,2,3]triazin-4(3H)-one;
6-(4-(4-chlorophenoxy)phenyl)-3-((3-methyl-1,2,4-oxadiazol-5-yl)methyl)benzo[d][1,2,3]triazin-4(3H)-one;
3-(2-(pyrimidin-4-yl)ethyl)-6-(4-(trifluoromethoxy)phenyl)benzo[d][1,2,3]triazin-4(3H)-one;
3-(2-(pyrimidin-2-yl)ethyl)-6-(4-(trifluoromethoxy)phenyl)benzo[d][1,2,3]triazin-4(3H)-one;
3-((5-phenyl-1H-tetrazol-1-yl)methyl)-6-(4-(trifluoromethoxy)phenyl)benzo[d][1,2,3]triazin-4(3H)-one;
3-cyclopropyl-6-(4-(trifluoromethoxy)phenyl)benzo[d][1,2,3]triazin-4(3H)-one;
3-((4,5-dimethyloxazol-2-yl)methyl)-6-(4-(trifluoromethoxy)phenyl)benzo[d][1,2,3]triazin-4(3H)-one;
3-(pyrimidin-2-ylmethyl)-6-(4-(trifluoromethoxy)phenyl)benzo[d][1,2,3]triazin-4(3H)-one;
3-((3-methylisoxazol-5-yl)methyl)-6-(4-(trifluoromethoxy)phenyl)benzo[d][1,2,3]triazin-4(3H)-one;
3-((5-methylisoxazol-3-yl)methyl)-6-(4-(trifluoromethoxy)phenyl)benzo[d][1,2,3]triazin-4(3H)-one;
3-((2H-benzo[d][1,2,3]triazol-2-yl)methyl)-6-(4-(trifluoromethoxy)phenyl)benzo[d][1,2,3]triazin-4(3H)-one;
3-(2-(1H-pyrazol-1-yl)ethyl)-6-(4-(4-chlorophenoxy)phenyl)benzo[d][1,2,3]triazin-4(3H)-one;
3-(2-(pyrimidin-2-yloxy)ethyl)-6-(4-(trifluoromethoxy)phenyl)benzo[d][1,2,3]triazin-4(3H)-one;
1-(4-oxo-6-(4-(trifluoromethoxy)phenyl)benzo[d][1,2,3]triazin-3 (4H)-yl)cyclopropanecarbonitrile;
3-((1-((2-methyl-1H-imidazol-1-yl)methyl)cyclopropyl)methyl)-6-(4-(trifluoromethoxy)phenyl)benzo[d][1,2,3]triazin-4(3H)-one;
2-(2-(4-oxo-6-(4-(trifluoromethoxy)phenyl)benzo[d][1,2,3]triazin-3 (4H)-yl)ethoxy)pyrimidine-4-carbonitrile;
3-(piperidin-4-yl)-6-(4-(trifluoromethoxy)phenyl)benzo[d][1,2,3]triazin-4(3H)-one;
3-(1-(pyrimidin-2-yl)piperidin-4-yl)-6-(4-(trifluoromethoxy)phenyl)benzo[d][1,2,3]triazin-4(3H)-one;
3-((1-(morpholinomethyl)cyclopropyl)methyl)-6-(4-(trifluoromethoxy)phenyl)benzo[d][1,2,3]triazin-4(3H)-one;
3-(2-oxo-2-(4-(primidin-2-yl)piperazin-1-yl)ethyl)-6-(4-(trifluoromethoxy)phenyl)benzo[d][1,2,3]triazin-4(3H)-one;
3-benzyl-6-(4-(trifluoromethoxy)phenyl)benzo[d][1,2,3]triazin-4(3H)-one;
3-((4,6-dimethoxypyrimidin-2-yl)methyl)-6-(4-(trifluoromethoxy)phenyl)benzo[d][1,2,3]triazin-4(3H)-one;
3-((5-(pyridin-2-yl)isoxazol-3-yl)methyl)-6-(4-(trifluoromethoxy)phenyl)benzo[d][1,2,3]triazin-4(3H)-one;
1-(4-(4-oxo-3-(2-(pyrimidin-2-yloxy) ethyl)-3,4-dihydrobenzo[d][1,2,3]triazin-6-yl)phenyl)cyclopropanecarbonitrile;
2-(2-(4-oxo-6-(4-(trifluoromethoxy)phenyl)benzo[d][1,2,3]triazin-3 (4H)-yl)ethoxy)pyrimidine-5-carbonitrile;
6-(4-(trifluoromethoxy)phenyl)-3-(2-(3-(trifluoromethyl)-1H-pyrazol-1-yl)ethyl)benzo[d][1,2,3]triazin-4 (3H)-one;
3-(1-(3-(pyrimidin-2-yl)-1,2,4-oxadiazol-5-yl)ethyl)-6-(4-(trifluoromethoxy)phenyl)benzo[d][1,2,3]triazin-4(3H)-one;
3-((5-(pyridin-2-yl)-1,2,4-oxadiazol-3-yl)methyl)-6-(4-(trifluoromethoxy)phenyl)benzo[d][1,2,3]triazin-4(3H)-one;
methyl 1-((4-oxo-6-(4-(trifluoromethoxy)phenyl)benzo[d][1,2,3]triazin-3 (4H)-yl)methyl)cyclopropanecarboxylate;
3-(pyrimidin-2-ylmethoxy)-6-(4-(trifluoromethoxy)phenyl)benzo[d][1,2,3]triazin-4(3H)-one;
3-((1-((2-ethyl-1H-imidazol-1-yl)methyl)cyclopropyl)methyl)-6-(4-(trifluoromethoxy)phenyl)benzo[d][1,2,3]triazin-4(3H)-one;
3-((1-((1H-imidazol-1-yl)methyl)cyclopropyl)methyl)-6-(4-(trifluoromethoxy)phenyl)benzo[d][1,2,3]triazin-4(3H)-one;
3-(pyridin-3-ylmethoxy)-6-(4-(trifluoromethoxy)phenyl)benzo[d][1,2,3]triazin-4(3H)-one;
3-(2-(4-(5-cyclopropyl-1,2,4-oxadiazol-3-yl)pyrimidin-2-yloxy)ethyl)-6-(4-(trifluoromethoxy)phenyl)benzo[d][1,2,3]triazin-4(3H)-one;
3-((1-(pyrrolidin-1-ylmethyl)cyclopropyl)methyl)-6-(4-(trifluoromethoxy)phenyl)benzo[d][1,2,3]triazin-4(3H)-one;
3-((1-((3,5-dimethyl-1H-pyrazol-1-yl)methyl)cyclopropyl)methyl)-6-(4-(trifluoromethoxy)phenyl)benzo[d][1,2,3]triazin-4(3H)-one;
6-(4-(4-chlorophenoxy)phenyl)-3-(2-oxo-2-(4-(pyrimidin-2-yl)piperazin-1-yl)ethyl)benzo[d][1,2,3]triazin-4(3H)-one;
3-((5-cyclopropyl-1,3,4-thiadiazol-2-yl)methyl)-6-(4-(trifluoromethoxy)phenyl)benzo[d][1,2,3]triazin-4(3H)-one;
3-((5-cyclopropyl-1,3,4-oxadiazol-2-yl)methyl)-6-(4-(trifluoromethoxy)phenyl)benzo[d][1,2,3]triazin-4(3H)-one;
3-(1-(2,2,2-trifluoroethyl)piperidin-4-yl)-6-(4-(trifluoromethoxy)phenyl)benzo[d][1,2,3]triazin-4(3H)-one;
ethyl 4-oxo-3-(4-oxo-6-(4-(trifluoromethoxy)phenyl)benzo[d][1,2,3]triazin-3(4H)-yl)piperidine-1-carboxylate;
6(4-cyclopropylphenyl)-3-((3-methyl-1,2,4-oxadiazol-5-yl)methyl)benzo[d][1,2,3]triazin-4(3H)-one;
3-((1-(hydroxymethyl)cyclopropyl)methyl)-6-(4-(trifluoromethoxy)phenyl)benzo[d][1,2,3]triazin-4(3H)-one;
3-(1-(3-cyclopropyl-1,2,4-oxadiazol-5-yl)ethyl)-6-(4-(trifluoromethoxy)phenyl)benzo[d][1,2,3]triazin-4(3H)-one;
3-((1-((pyrimidin-2-yloxy)methyl)cyclopropyl)methyl)-6-(4-(trifluoromethoxy)phenyl)benzo[d][1,2,3]triazin-4(3H)-one;
3-(2,2-dimethyl-3-(pyrimidin-2-yloxy)propyl)-6-(4-(trifluoromethoxy)phenyl)benzo[d][1,2,3]triazin-4(3H)-one;
3-((2-methyloxazol-5-yl)methyl)-6-(4-(trifluoromethoxy)phenyl)benzo[d][1,2,3]triazin-4(3H)-one;
3-((5-methyloxazol-2-yl)methyl)-6-(4-(trifluoromethoxy)phenyl)benzo[d][1,2,3]triazin-4(3H)-one;
3-((4-methyloxazol-2-yl)methyl)-6-(4-(trifluoromethoxy)phenyl)benzo[d][1,2,3]triazin-4(3H)-one;
3-((2-cyclobutyloxazol-4-yl)methyl)-6-(4-(trifluoromethoxy)phenyl)benzo[d][1,2,3]triazin-4(3H)-one;
3-((2-methyloxazol-4-yl)methyl)-6-(4-(trifluoromethoxy)phenyl)benzo[d][1,2,3]triazin-4(3H)-one;
3-((2-cyclopropyloxazol-4-yl)methyl)-6-(4-(trifluoromethoxy)phenyl)benzo[d][1,2,3]triazin-4(3H)-one;
3-((5-methyl-1,2,4-oxadiazol-3-yl)methyl)-6-(6-(2,2,2-trifluoroethoxy)pyridin-3-yl)benzo[d][1,2,3]triazin-4(3H)-one;
3-(2-(pyrimidin-2-yloxy)ethyl)-6-(6-(trifluoromethyl)pyridin-3-yl)benzo[d][1,2,3]triazin-4(3H)-one;
6-(2-(piperidin-1-yl)pyrimidin-5-yl)-3-(2-(pyridin-2-yloxy)ethyl)benzo[d][1,2,3]triazin-4(3H)-one;
3-(2-(pyrimidin-2-yloxy)ethyl)-6-((4-(trifluoromethoxy)phenyl)ethynyl)benzo[d][1,2,3]triazin-4(3H)-one;
2-((3-methyl-1,2,4-oxadiazol-5-yl)methyl)-7-(4-(trifluoromethoxy)phenyl)phthalazin-1(2E1)-one;
2-((5-methyl-1,2,4-oxadiazol-3-yl)methyl)-7-(4-(trifluoromethoxy)phenyl)phthalazin-1(2E1)-one;
2-(pyrimidin-2-ylmethyl)-7-(4-(trifluoromethoxy)phenyl)phthalazin-1(2H)-one
2-benzyl-7-(4-(trifluoromethoxy)phenyl)phthalazin-1(2H)-one;
2-phenethyl-7-(4-(trifluoromethoxy)phenyl)phthalazin-1(2H)-one;
2-(2-(1H-pyrazol-1-yl)ethyl)-7-(4-(trifluoromethoxy)phenyl)phthalazin-1(2H)-one;
2-(2-(1H-pyrrol-1-yl)ethyl)-7-(4-(trifluoromethoxy)phenyl)phthalazin-1(2H)-one;
2-((4-methyl-1,2,5-oxadiazol-3-yl)methyl)-7-(4-(trifluoromethoxy)phenyl)phthalazin-1(2H)-one;
6-((1-oxo-7-(4-(trifluoromethoxy)phenyl)phthalazin-2(1H)-yl)methyl)picolinonitrile;
2-((2-bromopyridin-3-yl)methyl)-7-(4-(trifluoromethoxy)phenyl)phthalazin-1(2H)-one;
2-(2-(3-methyl-1H-pyrazol-1-yl)ethyl)-7-(4-(trifluoromethoxy)phenyl)phthalazin-1(2H)-one;
2-(2-(6-methylpyridin-2-yl)ethyl)-7-(4-(trifluoromethoxy)phenyl)phthalazin-1(2H)-one;
2-((4,6-dimethoxypyrimidin-2-yl)methyl)-7-(4-(trifluoromethoxy)phenyl)phthalazin-1(2H)-one;
2-((2-cyclopropylpyridin-3-yl)methyl)-7-(4-(trifluoro methoxy)phenyl)phthalazin-1(2H)-one;
2-((4,6-dimethylpyrimidin-2-yl)methyl)-7-(4-(trifluoromethoxy)phenyl)phthalazin-1(2H)-one;
2-((4-cyclopropylpyrimidin-2-yl)methyl)-7-(4-(trifluoromethoxy)phenyl)phthalazin-1(2H)-one;
2-(2-(3,5-dimethyl-1,1-pyrazol-1-yl)ethyl)-7-(4-(trifluoromethoxy)phenyl)phthalazin-1(2H)-one;
2-(2-(1-methyl-1H-benzo[d]imidazol-2-yl)ethyl)-7-(4-(trifluoromethoxy)phenyl)phthalazin-1(2H)-one;
2-(2-(1H-1,2,4-triazol-1-yl)ethyl)-7-(4-(trifluoromethoxy)phenyl)phthalazin-1(2H)-one;
2-((4-(cyclopropylmethoxy)pyrimidin-2-yl)methyl)-7-(4-(trifluoromethoxy)phenyl)phthalazin-1(2H)-one;
2-(2-(pyrimidin-2-yloxy)ethyl)-7-(4-(trifluoromethoxy)phenyl)phthalazin-1(2H)-one;
2-(2-(4-cyclopropylpyrimidin-2-yloxy)ethyl)-7-(4-(trifluoromethoxy)phenyl)phthalazin-1(2H)-one;
2-((4-methoxypyrimidin-2-yl)methyl)-7-(4-(trifluoromethoxy)phenyl)phthalazin-1(2H)-one;
2-(2-(4-bromo-1H-pyrazol-1-yl)ethyl)-7-(4-(trifluoromethoxy)phenyl)phthalazin-1(2H)-one;
2-(2-(5-methyl-1H-pyrazol-1-yl)ethyl)-7-(4-(trifluoromethoxy)phenyl)phthalazin-1(2H)-one;
2-(2-(4-(2-methoxypyrimidin-5-yl)-1H-pyrazol-1-yl)ethyl)-7-(4-(trifluoromethoxy)phenyl)phthalazin-1(2H)-one;
2-((5-chloropyrimidin-2-yl)methyl)-7-(4-(trifluoromethoxy)phenyl)phthalazin-1(2H)-one;
2-(2-(pyrimidin-4-yl)ethyl)-7-(4-(trifluoromethoxy)phenyl)phthalazin-1(2H)-one;
2-(2-(5-chloropyrimidin-2-yloxy)ethyl)-7-(4-(trifluoromethoxy)phenyl)phthalazin-1(2H)-one;
2-(2-(1H-pyrazol-1-yl)propyl)-7-(4-(trifluoromethoxy)phenyl) phthalazin-1(2H)-one;
2-(2-(pyrazin-2-yloxy)ethyl)-7-(4-(trifluoromethoxy)phenyl)phthalazin-1(2H)-one;
2-(2-(pyridin-2-yloxy)ethyl)-7-(4-(trifluoromethoxy)phenyl)phthalazin-1(2H)-one;
2-((5-(pyridin-2-yl) isoxazol-3-yl)methyl)-7-(4-(trifluoromethoxy)phenyl)phthalazin-1(2H)-one;
3-((4-methyl-1,2,5-oxadiazol-3-yl)methyl)-6-(4-(trifluoromethoxy)phenyl)quinazolin-4(3H)-one;
3-((3-methyl-1,2,4-oxadiazol-5-yl)methyl)-6-(4-(trifluoromethoxy)phenyl)quinazolin-4(3H)-one;
3-((5-methyl-1,2,4-oxadiazol-3-yl)methyl)-6-(4-(trifluoromethoxy)phenyl)quinazolin-4(3H)-one;
3-(pyridin-2-ylmethyl)-6-(4-(trifluoromethyl)phenyl)-2H-benzo[e][1,3]oxazin-4(3H)-one;
3-(pyridin-2-ylmethyl)-6-(4-(trifluoromethoxy)phenyl)-2H-benzo[e][1,3]oxazin-4(3H)-one;
2-methyl-3-(pyrimidin-2-ylmethyl)-6-(4-(trifluoromethoxy)phenyl)-2H-benzo[e][1,3]oxazin-4(3H)-one;
2,2-dimethyl-3-(pyridin-2-ylmethyl)-6-(4-(trifluoromethoxy)phenyl)-2H-benzo[e][1,3]oxazin-4(3H)-one;
6-(2-fluoro-4-(trifluoromethyl)phenyl)-3-(pyrimidin-2-ylmethyl)-2H-benzo[e][1,3]oxazin-4(3H)-one;
3-(pyrimidin-2-ylmethyl)-6-(4-(trifluoromethyl)phenyl)-2H-benzo[e][1,3]oxazin-4(3H)-one;
3-(pyrimidin-2-ylmethyl)-6-(4-(trifluoromethoxy)phenyl)-2H-benzo[e][1,3]oxazin-4(3H)-one;
3-benzyl-6-(4-(trifluoromethyl)phenyl)-2H-benzo[e][1,3]oxazin-4(3H)-one;
3-benzyl-6-(2-fluoro-4-(trifluoromethyl)phenyl)-2H-benzo[e][1,3]oxazin-4(3H)-one;
3-benzyl-6-(4-(trifluoromethoxy)phenyl)-2H-benzo[e][1,3]oxazin-4(3H)-one;
3-(pyridin-2-ylmethyl)-6-(4-(trifluoromethoxy)phenyl)-2H-benzo[e][1,3]thiazin-4(3H)-one;
3-(pyrimidin-2-ylmethyl)-6-(4-(trifluoromethoxy)phenyl)-2H-benzo[e][1,3]thiazin-4(3H)-one;
3-(pyridin-2-ylmethyl)-6-(4-(trifluoromethyl)phenyl)-2H-benzo[e][1,3]thiazin-4(3H)-one;
3-(pyrimidin-2-ylmethyl)-6-(4-(trifluoromethyl)phenyl)-2H-benzo[e][1,3]thiazin-4(3H)-one;
3-(2-chlorobenzyl)-6-(4-(trifluoromethyl)phenyl)-2H-benzo[e][1,3]thiazin-4(3H)-one;
34(3-fluoropyridin-2-yl)methyl)-6-(4-(trifluoromethyl)phenyl)-2H-benzo[e][1,3]thiazin-4(3H)-one;
3-((3-fluoropyridin-2-yl)methyl)-6-(4-(trifluoromethoxy)phenyl)-2H-benzo[e][1,3]thiazin-4(3H)-one;
2-(pyrimidin-2-ylmethyl)-7-(4-(trifluoromethoxy)phenyl)-3,4-dihydroisoquinolin-1(2H)-one;
2-(pyridin-2-ylmethyl)-7-(4-(trifluoromethoxy)phenyl)-3,4-dihydroisoquinolin-1(2H)-one;
2-(pyrimidin-2-ylmethyl)-7-(4-(trifluoromethyl)phenyl)-3,4-dihydroisoquinolin-1(2H)-one;
2-(pyrimidin-2-ylmethyl)-7-(4-(trifluoromethyl)phenyl)ethynyl)-3,4-dihydroisoquinolin-1(2H)-one;
2-(pyrimidin-2-ylmethyl)-7-(4-(trifluoromethoxy)phenyl)ethynyl)-3,4-dihydroisoquinolin-1(2H)-one;
pyridin-2-yl(7-(4-(trifluoromethyl)phenyl)-3,4-dihydroisoquinolin-2(1H)-yl)methanone;
pyrimidin-2-yl(7-(4-(trifluoromethyl)phenyl)-3,4-dihydroisoquinolin-2(1H)-yl)methanone;
pyrimidin-2-yl(7-(4-(trifluoromethoxy)phenyl)-3,4-dihydroisoquinolin-2(1H)-yl)methanone;
pyridazin-3-yl(7-(4-(trifluoromethyl)phenyl)-3,4-dihydroisoquinolin-2(1H)-yl)methanone;
(7-(2-fluoro-4-(trifluoromethyl)phenyl)-3,4-dihydroisoquinolin-2(1H)-yl)(pyrimidin-2-yl)methanone;
(7-(4-chloro-2-fluorophenyl)-3,4-dihydroisoquinolin-2(1H)-yl)(pyrimidin-2-yl)methanone;
(7-(4-chloro-3-fluorophenyl)-3,4-dihydroisoquinolin-2(1H)-yl)(pyrimidin-2-yl)methanone;
(3-fluoropyridin-2-yl)(7-(4-(trifluoromethyl)phenyl)-3,4-dihydroisoquinolin-2(1H)-yl)methanone;
(7-(4-(trifluoromethyl)phenyl)-3,4-dihydroisoquinolin-2(1H)-yl)(1,3,5-trimethyl-1H-pyrazol-4-yl)methanone;
(1-isopropyl-1H-pyrazol-4-yl)(7-(4-(trifluoromethyl)phenyl)-3,4-dihydroisoquinolin-2(H)-yl)methanone;
(1,3-dimethyl-1H-pyrazol-4-yl)(7-(4-(trifluoromethyl)phenyl)-3,4-dihydroisoquinolin-2(1H)-yl)methanone;
2-(pyridin-2-yl)-1-(7-(4-(trifluoromethyl)phenyl)-3,4-dihydroisoquinolin-2(1H)-yl)ethanone;
2-(pyrimidin-2-yl)-1-(7-(4-(trifluoromethyl)phenyl)-3,4-dihydroisoquinolin-2(1H)-yl)ethanone;
(2-isopropylpyrimidin-4-yl)(7-(4-(trifluoromethyl)phenyl)-3,4-dihydroisoquinolin-2(1H)-yl)methanone;
pyrimidin-4-yl(7-(4-(trifluoromethyl)phenyl)-3,4-dihydroisoquinolin-2(1H)-yl)methanone;
pyrimidin-5-yl(7-(4-(trifluoromethyl)phenyl)-3,4-dihydroisoquinolin-2(1H)-yl)methanone;
(2-amino-6-methylpyrimidin-4-yl)(7-(4-(trifluoromethyl)phenyl)-3,4-dihydroisoquinolin-2(1H)-yl)methanone;
(1H-pyrazol-5-yl)(7-(4-(trifluoromethyl)phenyl)-3,4-dihydroisoquinolin-2(1H)-yl)methanone;
(1-methyl-1H-imidazol-4-yl)(7-(4-(trifluoromethyl)phenyl)-3,4-dihydroisoquinolin-2(1H)-yl)methanone;
N-benzyl-7-(4-(trifluoromethyl)phenyl)-3,4-dihydroisoquinoline-2(1H)-carboxamide;
N-phenyl-7-(4-(trifluoromethyl)phenyl)-3,4-dihydroisoquinoline-2(1H)-carboxamide;
N-cyclopropyl-7-(4-(trifluoromethyl)phenyl)-3,4-dihydroisoquinoline-2(1H)-carboxamide;
N-(furan-2-ylmethyl)-7-(4-(trifluoromethyl)phenyl)-3,4-dihydroisoquinoline-2(1H)-carboxamide;
N-methyl-N-phenyl-7-(4-(trifluoromethyl)phenyl)-3,4-dihydroisoquinoline-2(1H)-carboxamide;
morpholino (7-(4-(trifluoromethyl)phenyl)-3,4-dihydroisoquinolin-2(1H)-yl)methanone;
pyrrolidin-1-yl(7-(4-(trifluoromethyl)phenyl)-3,4-dihydroisoquinolin-2(1H)-yl)methanone;
(1-methyl-1H-imidazol-5-yl)(7-(4-(trifluoromethyl)phenyl)-3,4-dihydroisoquinolin-2(1H)-yl)methanone;
(1H-imidazol-2-yl)(7-(4-(trifluoromethyl)phenyl)-3,4-dihydroisoquinolin-2(1H)-yl)methanone;
(4-fluoro-1H-imidazol-5-yl)(7-(4-(trifluoromethyl)phenyl)-3,4-dihydroisoquinolin-2(1H)-yl)methanone;
N-cyclopentyl-7-(4-(trifluoromethyl)phenyl)-3,4-dihydroisoquinoline-2(1H)-carboxamide;
(1-methyl-1H-imidazol-2-yl)(7-(4-(trifluoromethyl)phenyl)-3,4-dihydroisoquinolin-2(1H)-yl)methanone;
azetidin-1-yl(7-(4-(trifluoromethyl)phenyl)-3,4-dihydroisoquinolin-2(1H)-yl)methanone
N-(pyrimidin-2-ylmethyl)-7-(4-(trifluoromethyl)phenyl)-3,4-dihydroisoquinoline-2(1H)-carboxamide;
(3-methylpyrrolidin-1-yl)(7-(4-(trifluoromethyl)phenyl)-3,4-dihydroisoquinolin-2(1H)-yl)methanone;
(3-hydroxypyrrolidin-1-yl)(7-(4-(trifluoromethyl)phenyl)-3,4-dihydroisoquinolin-2(1H)-yl)methanone;
(3,3-difluoro azetidin-1-yl)(7-(4-(trifluoromethyl)phenyl)-3,4-dihydroisoquinolin-2(1H)-yl)methanone;
(3-(pyridin-3-yloxy)azetidin-1-yl)(7-(4-(trifluoromethyl)phenyl)-3,4-dihydroisoquinolin-2(1H)-yl)methanone;
(3-fluoropyrrolidin-1-yl)(7-(4-(trifluoromethyl)phenyl)-3,4-dihydroisoquinolin-2(1H)-yl)methanone;
(3-fluoroazetidin-1-yl)(7-(4-(trifluoromethyl)phenyl)-3,4-dihydroisoquinolin-2(1H)-yl)methanone;
2-(1-methyl-1H-imidazol-4-ylsulfonyl)-7-(4-(trifluoromethyl)phenyl)-1,2,3,4-tetrahydroisoquinoline;
(R)-(3-(hydroxymethyl)pyrrolidin-1-yl)(7-(4-(trifluoromethyl)phenyl)-3,4-dihydroisoquino-2(1H)-yl)methanone;
(S)-(2-(hydroxymethyl)pyrrolidin-1-yl)(7-(4-(trifluoromethyl)phenyl)-3,4-dihydroisoquinolin-2(1H)-yl)methanone;
(3-(methyl sulfonyl)azetidin-1-yl)(7-(4-(trifluoromethyl)phenyl)-3,4-dihydroisoquinolin-2(1H)-yl)methanone;
((2R,5R)-2,5-dimethylpyrrolidin-1-yl)(7-(4-(trifluoromethyl)phenyl)-3,4-dihydroisoquinolin-2(1H)-yl)methanone;
((2R,5 S)-2,5-dimethylpyrrolidin-1-yl)(7-(4-(trifluoromethyl)phenyl)-3,4-dihydroisoquinolin-2(1H)-yl)methanone;
(3-methylazetidin-1-yl)(7-(4-(trifluoromethyl)phenyl)-3,4-dihydroisoquinolin-2(1H)-yl)methanone;
(3-hydroxyazetidin-1-yl)(7-(4-(trifluoromethyl)phenyl)-3,4-dihydroisoquinolin-2(1H)-yl)methanone;
(3-amino-1H-1,2,4-triazol-5-yl)(7-(4-(trifluoromethyl)phenyl)-3,4-dihydroisoquinolin-2(1H)-yl)methanone;
(3-hydroxy-3-methylazetidin-1-yl)(7-(4-(trifluoromethyl)phenyl)-3,4-dihydroisoquinolin-2(1H)-yl)methanone;
(3-(hydroxymethyl)azetidin-1-yl)(7-(4-(trifluoromethyl)phenyl)-3,4-dihydroisoquinolin-2(1H)-yl)methanone;
(R)-tert-butyl 2-(7-(4-(trifluoromethyl)phenyl)-1,2,3,4-tetrahydroisoquinoline-2-carbonyl)pyrrolidine-1-carboxylate;
(1-phenyl-1H-1,2,3-triazol-5-yl)(7-(4-(trifluoromethyl)phenyl)-3,4-dihydroisoquinolin-2(1H)-yl)methanone;
ethyl 2-(4-(7-(4-(trifluoromethyl)phenyl)-1,2,3,4-tetrahydroisoquinoline-2-carbonyl)-1H-1,2,3-triazol-1-yl)acetate;
pyrrolidin-1-yl(7-(4-(trifluoromethoxy)phenyl)-3,4-dihydroisoquinolin-2(1H)-yl)methanone; and
N-(pyrimidin-2-ylmethyl)-7-(4-(trifluoromethoxy)phenyl)-3,4-dihydroisoquinoline-2(1H)-carboxamide;
   or a pharmaceutically acceptable salt, ester, stereoisomer, prodrug or tautomer thereof.

Compounds of formula III are disclosed in U.S. patent application US2012/0289493A1 published Nov. 15, 2012, the entirety of which is incorporated herein by reference.

### Dosing

It is contemplated that a late I_{Na} inhibitor or pharmaceutically acceptable salt thereof and a CAMK II inhibitor or pharmaceutically acceptable salt thereof will both be administered in a therapeutically effective amount. In some embodiments, the late I_{Na} inhibitor is administered in a dose that is less than currently used, disclosed, estimated or approved for use, and the CAMK II inhibitor is administered at a standard therapeutically effective dose to achieve therapeutic effectiveness due to the synergistic effect of the combination of late I_{Na} inhibitor and CAMK II inhibitors. In other embodiment, the late I_{Na} inhibitor is administered at a less-than-standard therapeutic dose and the CAMK II inhibitor is administered in a standard therapeutically effective dose. In still other embodiments, both the late I_{Na} inhibitor and the CAMK II inhibitor are administered in less than standard therapeutic doses. In yet other embodiments, both the late I_{Na} inhibitor and the CAMK II inhibitor are administered at their standard doses or at doses determined by a qualified treating care giver.

As mentioned above, the CAMK inhibitor e.g. AIP or KN-93 may be administered in a synergistically therapeutic amount ranging from about 1 mg to about 2000 mg daily or from about 500 mg to about 1000 mg daily. Other dose ranges of CAMK inhibitors include 1 mg to 5 mg; 5 mg to 100 mg; and 200 mg to 500 mg. Similarly, doses of the late I_{Na} inhibitor, e.g. ranolazine, GS967, compound of Formula I, II, or III may be administered in an amount that provides synergy with the amount of CAMK inhibitor administered. Doses of late I_{Na} inhibitor may range from about 1 mg to about 2000 mg. In other embodiments, doses of late I_{Na} inhibitor may range from about 10 mg to about 1500 mg, from about 100 mg to about 1000 mg, from about 50 mg to about 500 mg or from about 5 mg to 200 mg.

A qualified care giver is in the best position to determine the appropriate dose for a given patient. The qualified care giver will take into consideration such factors as the age, weight, gender, patient history, presenting symptoms and their severity, co-presenting symptoms or diseases, frequency of administration, concomitant medications being taken by the patient, or whether a loading dose or a maintenance dose is required.

### Pharmaceutical Formulations

As mentioned above, a late I_{Na} inhibitor, e.g. ranolazine, GS-967, a compound of Formula I, II, or III, and a CAMK II inhibitor, e.g. KN-93 or AIP, may be co-administered, meaning that the two active ingredients may be formulated separately but administered at similar times (i.e., either simultaneously or sequentially, that is, together or one after the other). Co-administration also means that a late I_{Na} inhibitor e.g. ranolazine, GS-967, a compound of Formula I, II, or III and a CAMK II inhibitor e.g. KN-93 or AIP may be coformulated into a combined or fixed dosage unit. Accordingly, in one embodiment, the disclosure is directed to pharmaceutical formulations comprising a therapeutically effective amount of a CAMK II inhibitor or pharmaceutically acceptable salt thereof, a therapeutically effective amount of late I_{Na} inhibitor, and a pharmaceutically acceptable carrier.

In another embodiment, the formulation comprises a synergistically effective amount of a late I_{Na} inhibitor and a CAMK II inhibitor or pharmaceutically acceptable salt thereof. In certain embodiments, the formulations are formulated for either intravenous or oral administration.

In one embodiment, the two active ingredients are coformulated into a combined dosage unit. In still yet other embodiments, the two active ingredients are formulated separately for co-administration. In certain embodiments of the present disclosure, the late I_{Na} inhibitor and CAMK II inhibitor are coformulated into a combined dosage unit or unitary dosage form suitable for oral administration. In certain embodiments, ranolazine is formulated as a sustained release formulation. In certain embodiments, ranolazine is formulated for sustained release.

In one such embodiment, a pharmaceutically acceptable composition comprising late I_{Na} inhibitor is placed in a portion of the tablet which is separate from, but in contact with the portion of the tablet comprising a pharmaceutically acceptable composition of a CAMK II inhibitor. It will be understood that the unitary dosage form may comprise simply compressing the separate compositions of late I_{Na} inhibitor e.g. ranolazine and a CAMK II inhibitor, e.g. KN-93, into a multilayer tablet or conventionally processed into other conventional unitary dosage forms such as a capsules. The multilayer tablets and capsules suitable for use in the present disclosure may be fabricated using methods known in the art using standard machinery.

The tablets may comprise two layers, i.e. a first layer which comprises the late I_{Na} inhibitor formulated for immediate or sustained release, and a second layer which comprises the CAMK II inhibitor formulated for immediate or sustained release. For example, the multilayer tablet may comprise an inner layer and an outer layer, where the inner layer comprises the sustained release ranolazine formulation and where the outer layer comprises the immediate release or sustained release CAMK II inhibitor, e.g. KN-93 or AIP. In another embodiment, ranolazine and AIP or KN-93 are coformulated into a capsule, where the capsule allows for the immediate release or sustained release of AIP or KN-93 and the sustained release of ranolazine. For example, the capsule may contain granules of both a late I_{Na} inhibitor, e.g. ranolazine, GS-967, a compound of Formula I, II, or III, and a CAMK II inhibitor, e.g. KN-93 or AIP, where the granules have been formulated such that the KN-93 or AIP is available for immediate release or sustained release and the ranolazine, GS-967, Compound of formula I, II, or III is formulated for sustained release. Alternatively, the capsule may contain a liquid immediate release or sustained release formulation of KN-93, AIP or other CAMK II inhibitor and a solid sustained release formulation of ranolazine or other late I_{Na} inhibitor. However, such embodiments are exemplary and are not intended to limit the formulations of the present disclosure.

A multilayer tablet can be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active agent or dispersing agent. Molded tablets may be made by molding in a suitable machine a mixture of the powdered active ingredient moistened with an inert liquid diluent. The tablets may optionally be coated or scored. It is within the contemplation of the present disclosure toad an intermediate layer of fillers e.g. starch if it is desired to keep the layers of CAMK II inhibitor and the layer of late I_{Na} inhibitor separate prior to administration.

The tablets may contain one or more agents including sweetening agents, flavoring agents, coloring agents and preserving agents, in order to provide a palatable preparation. Tablets containing the active ingredients in admixture with non-toxic pharmaceutically acceptable excipients which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations contemplated by the present disclosure may also be for administration by injection including aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. Preventing the action of microorganisms can be brought about by use of various antibacterial and antifungal agents, for example, parabens, chlorobutanol, phenol, sorbic acid, thimerosal, and the like. Similar formulations are contemplated for separate but simultaneous or sequential administration of ranolazine or other late I_{Na} inhibitor and KN-93 or other CAMK Inhibitor.

Sterile injectable solutions are prepared by incorporating the component in the required amount in the appropriate solvent with various other ingredients as enumerated above, and as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The ideal forms of the apparatus for administration of the novel combinations for atrial fibrillation and other methods of the disclosure consist therefore of (1) either a syringe comprising 2 compartments containing the 2 active substances ready for use or (2) a kit containing two syringes ready for use.

In making pharmaceutical compositions that include a late I_{Na} inhibitor, e.g. ranolazine, and a CAMK II inhibitor, e.g. KN-93 or AIP, the active ingredients are usually diluted by an excipient or carrier and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the disclosure can be formulated so as to provide quick, sustained or delayed release of the active ingredient after administration to the patient by employing procedures known in the art. Sustained release formulations are generally preferred. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

The compositions are preferably formulated in a unit dosage form. The term “unit dosage forms” or “combined dosage unit” or “fixed dosage combination” refers to physically discrete units suitable as unitary dosages for human subjects and other mammals, each unit containing a predetermined quantity of the active materials calculated to produce the desired synergistic therapeutic effect as described herein, in association with a suitable pharmaceutical excipient (e.g., a tablet, capsule, ampoule). A tablet or capsule is an example of a fixed dose combination that is a solid giving rise to the term “solid fixed dose” or “fixed dose solid” combination. The active agents of the disclosure are effective over a wide dosage range and are generally administered in a pharmaceutically effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient(s) to form a solid pre-formulation composition containing a homogeneous mixture of a compound of the present disclosure. When referring to these pre-formulation compositions as homogeneous, it is meant that the active ingredients are dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. The co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Additional embodiments of the disclosure include kits comprising a therapeutically effective amount of a late I_{Na} inhibitor e.g. ranolazine and a CAMK II inhibitor e.g. KN-93, and or a pharmaceutically acceptable salt or salts thereof.

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### EXAMPLES

### Materials and Methods

### Animals

Male Sprague-Dawley rats (300-350 g) were purchased from Charles River Laboratories (Wilmington, Mass.). The use of animals was approved by the Animal Care and Use Committee of Gilead Sciences, Inc.

### Chemicals

Ranolazine and GS-967 (6-(4-(trifluoromethoxy)phenyl)-3-(trifluoromethyl)-[1,2,4]triazolo[4,3-a]pyridine) were obtained from Gilead Sciences (Foster City, Calif.). KBR7943, KN-93, KN-92, and autocamtide 2-related inhibitory peptide (AIP) were purchased from EMD Millipore Chemicals (Billerica, Mass.). Scrambled AIP was prepared by and purchased from GeneScript (Piscataway, N.J.). ATX-II was purchased from Sigma-Aldrich (St. Louis, Mo.).

### Recording of Right Atrial Contractile and Electrical Activity

Rats were anesthetized by intraperitoneal injection of 60 mg/kg ketamine and 8 mg/kg xylazine. The heart was rapidly removed and immersed in oxygenated Krebs-Henseleit buffer (in mmol/L: 118 NaCl, 4.8 KCl, 1.2 KH₂PO₄, 2.5 CaCl₂, 1.2, MgSO₄, 2 pyruvic acid, 5.5 glucose, 0.5 Na₂EDTA, and 25 NaHCO₃, pH 7.4). Rat right atria were quickly excised and suspended in a chamber of the Danish Myo Technologies (DMT) myograph containing 8 ml of oxygenated Krebs-Henseleit buffer. Mechanical and electrical changes were measured simultaneously in spontaneously beating atria. Isometric tension was recorded by a force transducer connected to a PowerLab data acquisition system (AD Instruments, Colorado Springs, Colo.). A bipolar electrode was attached to the surface of the atrium to record an electrogram. After stabilization, the atria were preincubated with a late I_{Na} inhibitor (0.1 μmol/L GS967 or 3 μmol/L ranolazine), a CaMKII inhibitor ((0.3 μmol/L AIP or 0.3 μmol/L KN-93) or their combination for 30 min, then treated with 50 nmol/L ATX-II to induce arrhythmias and diastolic dysfunction.

### Quantification of Arrhythmias

Isolated right atria beat spontaneously at a rate of 298+12 bpm. ATX-II induced premature beats, tachyarrhythmia and fibrillation in rat isolated right atria. Arrhythmias were recorded for 30 min following ATX-II treatment, and quantified using an arbitrary numerical grading of perceived severity as described previously (32) and shown in Table 1.

TABLE 1
Arrhythmia Scores

| Score | Type of Arrhythmias |
|---|---|
| 0 | <0-10 premature atrial beats (PABs) |
| 1 | 10-100 PABs |
| 2 | >100 PABs or 1-3 episodes of atrial tachycardia (AT) or both |
| 3 | 3-10 episodes of AT |
| 4 | >10 episodes of AT, or 1-2 episodes of AF |
| | (duration <10 sec) or both |
| 5 | 3-5 episodes of AF or >10 sec duration of AF |
| 6 | 6-10 episodes of AF or >30 sec duration of AF |
| 7 | >10 episodes of AF or >60 sec duration of AF |
| 8 | Stop beating |

### Western Blot Analysis

Western blot analysis of (please describe the sample) was performed. Antibodies directed against phospho-CaMKII at threonine 286 and GAPDH were purchased from Santa Cruz Biotechnology. An antibody directed against CaMKII delta was purchased from Abeam (Cambridge, Mass.). An antibody directed against phospho-PLB at threonine 17 was purchased from Badrilla (Leeds, UK). Secondary antibody was horseradish peroxidase (HRP)-linked goat anti-rabbit (1:1,000) (PerkinElmer Life and Analytical Sciences) or protein A/G-HRP (Thermo Fisher Scientific, Waltham, Mass.). Relative intensity of individual bands from Western blots was quantitated using Image software and normalized to GAPDH. The ratio for control was assigned a value of 1.

### Right Atrial Myocyte Preparation

Adult male rats were anesthetized and hearts were rapidly removed and perfused in a retrograde mode (Langendorff preparation) with a modified Tyrode solution containing (in mmol/L): NaCl (135), KCl (4.6), MgCl₂ (1.1), NaH₂PO₄ (1) glucose (10), HEPES (10), pH 7.4. Hearts were perfused for 5 min with a nominal Ca²⁺-free Tyrode solution, then with a low Ca²⁺ (2 □mol/L) solution containing 0.896 mg/ml collagenase type II (Worthington, USA) and 0.68 mg/ml bovine serum albumin (BSA) for 8-11 min at 37° C. The right atria were excised from hearts and subjected to digestion in the presence of 0.35 mg/ml collagenase and 9.8 mg/ml BSA in a temperature-controlled rotating water bath, leading to graduate tissue dissociation. Single quiescent myocytes were selected for study.

### Intracellular Ca²⁺ and Na⁺ Measurement in Isolated Atrial Myocytes

Confocal microscopy experiments to measure changes in concentrations of intracellular Ca²⁺ or Na⁺ in isolated right atrial myocytes were performed using a LSM 5 PASCAL (Carl Zeiss, Germany) laser scanning confocal system equipped with a Zeiss Plan-Apochromat 63×1.4 numerical aperture oil immersion objective. The bath solution contained (in mmol/L): 140 NaCl, 5.4 KCl, 2.0 CaCl₂, 1 MgCl₂, 10 HEPES and 5.6 glucose, pH 7.3. Cells were loaded with the appropriate fluorescent dye (either 5 μmol/L fluo-4 AM

[Molecular Probes, USA], indo-1 AM [Molecular Probes] or 5 μmol/L Asante NaTRIUM Green 2 AM (Teflabs, USA) by incubation for 25 min at 23° C. in bath solution containing 1.0 mmol/L Ca²⁺, and 2.5 μmol/L pluronic acid (Molecular Probes). Fluo-4 was excited at 488 nm, and the fluorescence was acquired at wavelengths >505 nm in the line scan mode or XY mode of the confocal system. Asante NaTRIUM Green 2 was excited at 543 nm, and fluorescence was acquired at wavelengths >560 nm in the XY scan mode. Intracellular calcium concentrations were monitored in electrically stimulated cells loaded with indo-1 AM using an IonOptix spectrophotometer (IonOptix, Milton, Mass.). Indo-1 was excited by 365-nm light. Emitted fluorescence (405 nm and 485 nm) was detected by a photomultiplier tube. Experiments were performed at room temperature (23° C.) on the stage of an inverted microscope (Nikon, Tokyo, Japan), using a Plan Fluor 40× objective. Field stimulation (40 V, 1 Hz) was accomplished using an SD9 stimulator (Grass Products, Warwick, R¹). Fluorescent images were analyzed using ImageJ software (NIH) and Origin 8.1 (OriginLab, USA).

### Data Analysis

Data were expressed as mean±SEM and analyzed using one-way ANOVA with Newman-Keuls tests. All statistical analyses were performed using GraphPad Prism. Differences were considered significant when p<0.05.

### Results

Low concentrations of inhibitors of either late I_{Na} or CaMKII caused a modest inhibition of ATX-II-induced arrhythmias. As shown in Table 1, 3 μmol/L ranolazine, 100 nmol/L GS967, 300 nmol/L KN-93 and 300 nmol/L AIP individually attenuated ATX-II-induced arrhythmias by 28, 34, 23, and 19%, respectively. The combination of 3 mol/L ranolazine with 300 nmol/L KN-93 or AIP inhibited ATX-II-induced arrhythmias by 84% or 88%. Similarly, the combination of 100 nmol/L GS967 with 300 nmol/L KN-93 or AIP inhibited ATX-II-induced arrhythmias by 89% or 81% respectively. Either ranolazine or GS967 in combination with KN-93 or AIP generated a greater anti-arrhythmic effect than the calculated sum of individual effects of each compound, suggesting that a late I_{Na} inhibitor and a CaMKII inhibitor act synergistically to inhibit ATX-II-induced arrhythmias.

TABLE 1

| | measured | Calculated % |
|---|---|---|
| Group | % Inhibition | Inhibition (Σ) |
| 3 μM RAN | 23.8 ± 3.1 | |
| 0.1 μM GS-967 | 34.2 ± 4.8 | |
| 0.3 μM KN-93 | 24.1 ± 7.6 | |
| 0.3 μM AIP | 19.5 ± 2.6 | |
| Ran + KN-93 | 83.8 ± 3.0* | 47.9 ± 5.6 |
| Ran + AIP | 87.9 ± 4.1* | 43.3 ± 6.0 |
| GS967 + KN-93 | 89.2 ± 4.3* | 58.3 ± 5.8 |
| GS967 + AIP | 80.6 ± 4.0* | 53.7 ± 5.7 |

Three μmol/L ranolazine, 100 nmol/L GS967, 300 nmol/L KN-93 and AIP each attenuated an ATX-II-induced increase of diastolic tension by 10.4, 9.1, 8.4 and 9.5%, respectively, whereas the combination of ranolazine or GS967 with either KN-93 or AIP inhibited ATX-II-induced diastolic tension by 40-50%, which was also significantly greater than the calculated sum of individual effects of each compound (Table 2).

TABLE 2

| | Measured % | Calculated % |
|---|---|---|
| Group | Inhibition | Inhibition (Σ) |
| 3 μM RAN | 5.5 ± 1.5 | |
| 0.1 μM GS-967 | 9.1 ± 1.7 | |
| 0.3 μM KN-93 | 8.4 ± 1.8 | |
| 0.3 μM AIP | 9.5 ± 3.2 | |
| Ran + KN-93 | 40.9 ± 7.0* | 13.9 ± 2.0 |
| Ran + AIP | 40.2 ± 6.9* | 15.0 ± 4.2 |
| GS-967 + KN-93 | 48.4 ± 7.2* | 17.5 ± 3.0 |
| GS-967 + AIP | 45.5 ± 6.0* | 18.6 ± 3.5 |

Three μmol/L ranolazine, 100 nmol/L GS967, 300 nmol/L KN-93 and AIP alone failed to attenuate ATX-II-induced AF whereas the combination of 3 μmol/L ranolazine or 100 nmol/L GS967 with either 300 nmol/L KN-93 or 300 nmol/L AIP completely prevented ATX-II-induced AF in isolated rat right atria as shown in FIG. 1.

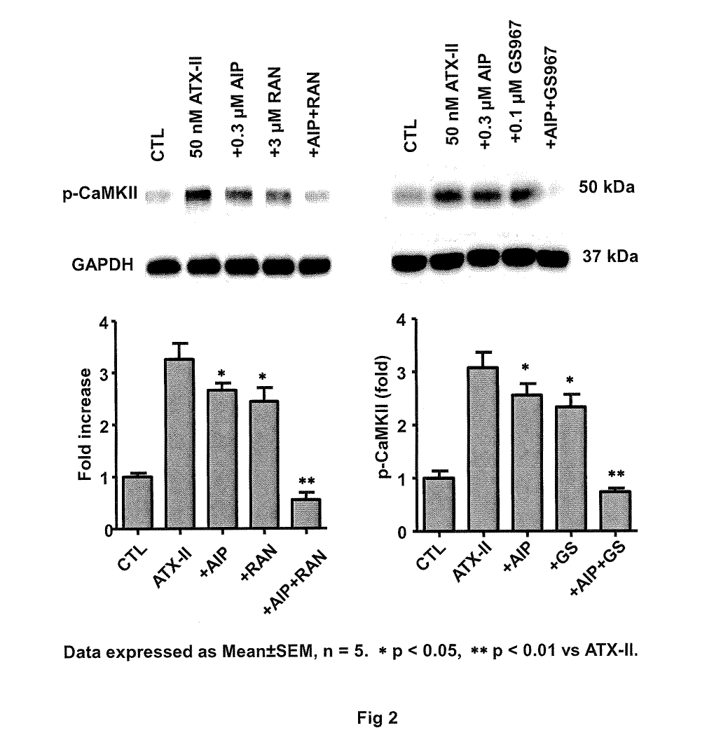

Three μmol/L ranolazine, 100 nmol/L GS967, 300 nmol/L KN-93 and 300 nmol/L AIP individually inhibited ATX-II-induced CaMKII phosphorylation by 20-30%. The combination of 3 μmol/L ranolazine with 300 nmol/L AIP resulted in a complete inhibition of ATX-II-induced CaMKII phosphorylation. Similarly, 100 nmol/L GS967 in combination with 300 nmol/L AIP also completely inhibited ATX-II-induced CaMKII phosphorylation as shown in FIG. 2. These data demonstrate the synergistic effects of a late I_{Na} inhibitor and a CaMKII inhibitor to attenuate ATX-II-induced increases of arrhythmias and diastolic tension.

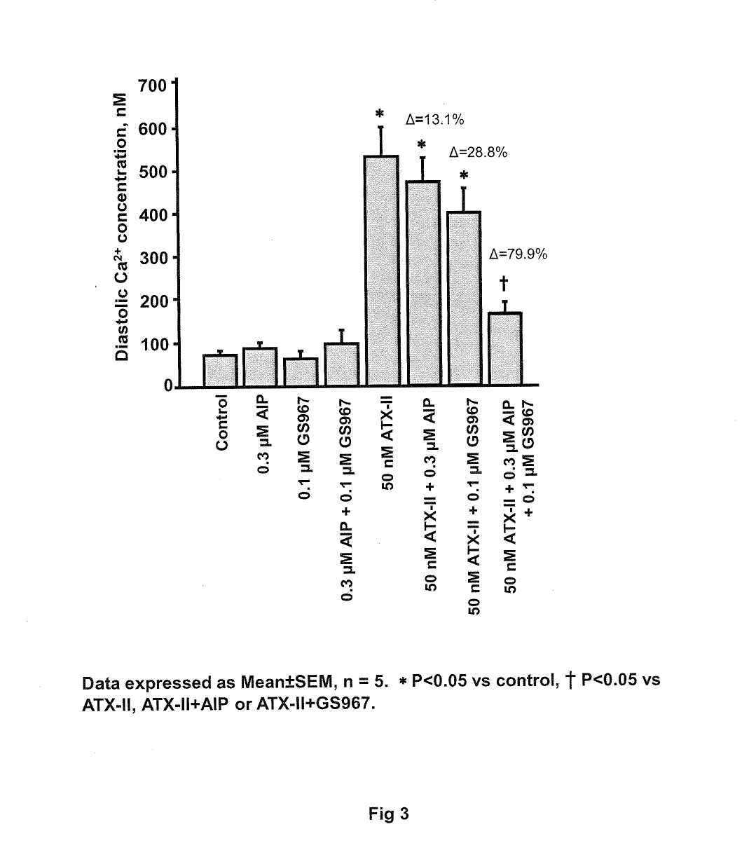

GS967 (100 nmol/L) and 300 nmol/L AIP individually inhibited the ATX-II-induced increase of diastolic Ca²⁺ by 13 and 29%, respectively, whereas the combination of both compounds caused an 80% inhibition (as shown in FIG. 3), which was much greater than the calculated sum of the individual inhibitions caused by each compound.

We claim:

1. A method for treating arrhythmias in a human patient comprising administering to the patient a therapeutically effective amount of a CAMK II inhibitor and a therapeutically effective amount of a late I_{Na} inhibitor.

2. The method according to claim 1 wherein the arrhythmias is atrial fibrillation or atrial flutter.

3. The method according to claim 1 wherein the CAMK inhibitor is AIP or KN-93.

4. The method according to claim 1 wherein the late I_{Na} inhibitor is ranolazine or GS-967.

5. The method according to claim 1 wherein the late I_{Na} inhibitor is a compound having the structure: 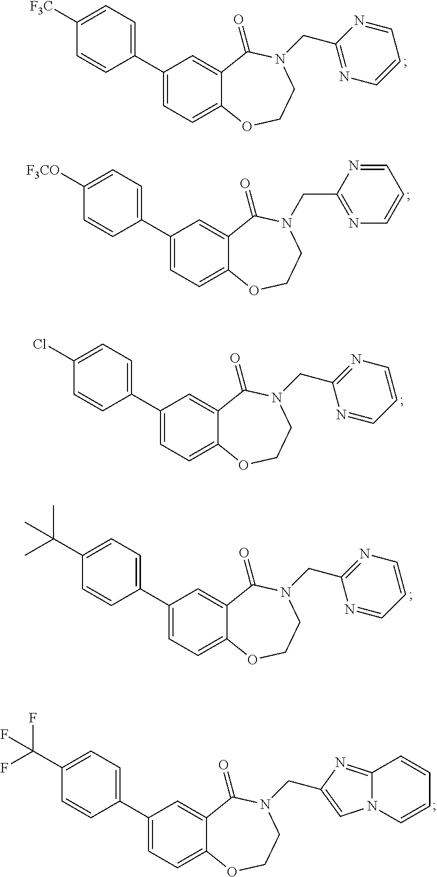 [SMILES: O=C1c2cc(-c3ccc(C(F)(F)F)cc3)ccc2OCCN1Cc1ncccn1 ; O=C1c2cc(-c3ccc(OC(F)(F)F)cc3)ccc2OCCN1Cc1ncccn1 ; O=C1c2cc(-c3ccc(Cl)cc3)ccc2OCCN1Cc1ncccn1 ; CC(C)(C)c1ccc(-c2ccc3c(c2)C(=O)N(Cc2ncccn2)CCO3)cc1 ; O=C1c2cc(-c3ccc(C(F)(F)F)cc3)ccc2OCCN1Cc1cn2ccccc2n1]
or a pharmaceutically acceptable salt thereof.

6. The method according to claim 1 wherein the therapeutically effective amount of a CAMK II inhibitor and therapeutically effective amount of a late I_{Na} inhibitor are administered simultaneously.

7. The method according to claim 1 wherein the therapeutically effective amount of a CAMK II inhibitor and therapeutically effective amount of a late I_{Na} inhibitor are administered separately.

8. The method according to claim 1 wherein the therapeutically effective amount of a CAMK II inhibitor and therapeutically effective amount of a late I_{Na} inhibitor are administered in a fixed dose solid combination.

9. The method according to claim 8 wherein the fixed dose combination is a tablet.

10. A method for treating heart failure in a human patient comprising administering to the patient a therapeutically effective amount of a CAMK II inhibitor and a therapeutically effective amount of a late I_{Na} inhibitor.

11. The method according to claim 10 wherein the heart failure is systolic heart failure.

12. The method according to claim 10 wherein the CAMK inhibitor is AIP or KN-93.

13. The method according to claim 10 wherein the late I_{Na} inhibitor is ranolazine or GS-967.

14. The method according to claim 10 wherein the late I_{Na} inhibitor is a compound having the structure: 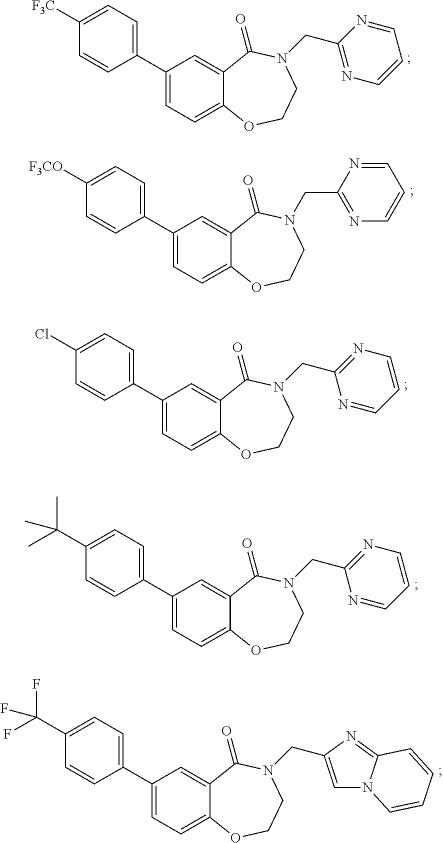 [SMILES: O=C1c2cc(-c3ccc(C(F)(F)F)cc3)ccc2OCCN1Cc1ncccn1 ; O=C1c2cc(-c3ccc(OC(F)(F)F)cc3)ccc2OCCN1Cc1ncccn1 ; O=C1c2cc(-c3ccc(Cl)cc3)ccc2OCCN1Cc1ncccn1 ; CC(C)(C)c1ccc(-c2ccc3c(c2)C(=O)N(Cc2ncccn2)CCO3)cc1 ; O=C1c2cc(-c3ccc(C(F)(F)F)cc3)ccc2OCCN1Cc1cn2ccccc2n1]
or a pharmaceutically acceptable salt thereof.

15. The method according to claim 10 wherein the therapeutically effective amount of a CAMK II inhibitor and therapeutically effective amount of a late I_{Na} inhibitor are administered simultaneously.

16. The method according to claim 10 wherein the therapeutically effective amount of a CAMK II inhibitor and therapeutically effective amount of a late I_{Na} inhibitor are administered separately.

17. The method according to claim 10 wherein the therapeutically effective amount of a CAMK II inhibitor and therapeutically effective amount of a late I_{Na} inhibitor are administered in a solid fixed dose combination.

18. The method according to claim 17 wherein the solid fixed dose combination is a tablet.

19. A pharmaceutical formulation comprising a therapeutically effective amount of a CAMK II inhibitor and therapeutically effective amount of a late Iₐ inhibitor and a pharmaceutically acceptable excipient.

20. The formulation according to claim 19 which is a solid.

21. The formulation according to claim 19 which is a liquid.

22. The formulation according to claim 19 which is an IV solution.
